SEQUENCE LISTING (22 sequences)

SEQ ID NO: 1 - protein, 26 aa, Artificial Sequence
  Synthetic Construct
KAQKAQAKQAKQAQKAQKAQAKQAKQ

SEQ ID NO: 2 - protein, 26 aa, Artificial Sequence
  Synthetic Construct
RAQRAQARQARQAQRAQRAQARQARQ

SEQ ID NO: 3 - protein, 15 aa, Artificial Sequence
  Synthetic Construct
KAKAKAKAKAKAKAK

SEQ ID NO: 4 - protein, 29 aa, Artificial Sequence
  Synthetic Construct
KAQAKAQAKAQAKAQAKAQAKAQAKAQAK

SEQ ID NO: 5 - protein, 26 aa, Artificial Sequence
  Synthetic Construct
KAQQAQAKQAQQAQKAQQAQAKQAQQ

SEQ ID NO: 6 - protein, 26 aa, Artificial Sequence
  Synthetic Construct
QAQKAQAQQAKQAQQAQKAQAQQAKQ

SEQ ID NO: 7 - protein, 26 aa, Artificial Sequence
  Synthetic Construct
KAQKAQAKQAKQAQKAQKAQAKQAKQ

SEQ ID NO: 8 - protein, 26 aa, Artificial Sequence
  Synthetic Construct
KTVKTVTKVTKVTVKTVKTVTKVTKV

SEQ ID NO: 9 - protein, 18 aa, Artificial Sequence
  Synthetic Construct
VYKVKTKVKTKVKTKVKT

SEQ ID NO: 10 - protein, 30 aa, Artificial Sequence
  Synthetic Construct
AQAYSKAQKAQAKQAKQAQKAQKAQAKAKQ

SEQ ID NO: 11 - protein, 31 aa, Artificial Sequence
  Synthetic Construct
AQAYARAQRAQARQARQAQRAQRAQARQARQ

SEQ ID NO: 12 - protein, 40 aa, Artificial Sequence
  Synthetic Construct
KAQKAQAKQAKQAQKAQKAQAKQAKQAQKAQKAQAKQAKQ

SEQ ID NO: 13 - protein, 40 aa, Artificial Sequence
  Synthetic Construct
RAQRAQARQARQAQRAQRAQARQARQAQRAQRAQARQARQ

SEQ ID NO: 14 - protein, 5 aa, Artificial Sequence
  Synthetic Construct
VSPSV

SEQ ID NO: 15 - protein, 10 aa, Artificial Sequence
  Synthetic Construct
VSPSVVSPSV

SEQ ID NO: 16 - protein, 5 aa, Artificial Sequence
  Synthetic Construct
GGSGG

SEQ ID NO: 17 - protein, 10 aa, Artificial Sequence
  Synthetic Construct
GGGGSGGGGS

SEQ ID NO: 18 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 263 aa, Artificial Sequence
  Synthetic Construct
APGGGRAQRAQARQARQAQRAQRAQARQARQVSPSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 266 aa, Artificial Sequence
  Synthetic Construct
APGGGSVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVSPSVRAQRAQARQARQAQRAQRAQARQARQ

SEQ ID NO: 21 - protein, 271 aa, Artificial Sequence
  Synthetic Construct
APGGGSVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRAQRAQARQARQAQRAQRAQARQARQ

SEQ ID NO: 22 - protein, 280 aa, Artificial Sequence
  Synthetic Construct
APGGGSVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVSPSVRAQRAQARQARQAQRAQRAQARQARQAQRAQRAQARQARQ

(19) United States
(12) Patent Application Publication
(10) Pub. No.: US 2024/0216556 A1
(43) Pub. Date: Jul. 4, 2024

(54) PEPTIDE-FC FUSIONS FOR TREATING AMYLOID DISORDERS

(71) Applicants: UNIVERSITY OF TENNESSEE RESEARCH FOUNDATION, Knoxville, TN (US); Attralus, Inc., San Francisco, CA (US)

(72) Inventors: Jonathan S. WALL, Knoxville, TN (US); James S. FOSTER, Knoxville, TN (US); Jaume PONS, San Francisco, CA (US)

(73) Assignees: UNIVERSITY OF TENNESSEE RESEARCH FOUNDATION, Knoxville, TN (US); Attralus, Inc., Burlingame, CA (US)

(21) Appl. No.: 18/289,032

(22) PCT Filed: May 4, 2022
(86) PCT No.: PCT/US2022/072112

§ 371 Date: Oct. 31, 2023

Related U.S. Application Data
(60) Provisional application No. 63/184,682, filed on May 5, 2021.
(60) Provisional application No. 63/186,605, filed on May 10, 2021.

(51) Int. Cl. A61K 51/10; A61K 49/00; A61P 25/28; C07K 7/06; C07K 14/00

(52) U.S. Cl. CPC A61K 51/1096; A61K 49/0058; A61P 25/28; C07K 7/06; C07K 14/00; C07K 2319/30

(57) ABSTRACT

Provided herein are amyloid-reactive peptide-Fc fusion proteins comprising an amyloid-reactive peptide linked to a human Fc region. Also provided herein are methods of treating amyloid-based diseases and identifying amyloid deposits by administering an amyloid-reactive peptide-Fc fusion protein comprising an amyloid-reactive peptide linked to a human Fc region.

### CROSS REFERENCE TO RELATED APPLICATIONS

This application claims priority to U.S. Provisional Application No. 63/184,682, filed May 5, 2021 and U.S. Provisional Application No. 63/186,605 filed May 10, 2021, the contents of each of which are hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165992000840SEQLIST.TXT, date recorded: May 4, 2022, size: 16,694 bytes).

### FIELD OF THE INVENTION

The present invention relates to amyloid-reactive peptide-Fc fusion proteins, methods of treating amyloid-related disorders by administering amyloid-reactive peptide-Fc fusion proteins, and methods of detecting amyloid using amyloid-reactive peptide-Fc fusion proteins.

### BACKGROUND OF THE INVENTION

Amyloidosis, is a broad group of diseases that belong to the group of conformational protein diseases that include other diseases such as AA amyloidosis, AL amyloidosis, AH amyloidosis, Aβ amyloidosis, ATTR amyloidosis, hATTR amyloidosis, ALect2 amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer.

Amyloidosis is a rare disease characterized by the presence of insoluble protein deposits with abnormal fibrillar conformation in tissues. Most often, it is fragments of serum precursor proteins that are the cause. Many organs can be affected by these extracellular deposits, called “amyloid substance”. The main organs affected by amyloid deposits are the kidney, the heart, the digestive tract, the liver, the skin, the peripheral nerve and the eye. The organs affected by this disease usually have a considerable volume. Ultimately, amyloidosis can affect all organs as well as the central nervous system so that there are many very varied symptoms.

Accordingly, there is a need for effective treatments for amyloidosis and amyloid-related diseases.

### SUMMARY OF THE INVENTION

In one aspect, provided herein is an amyloid-reactive peptide-Fc fusion protein comprising a first polypeptide and a second polypeptide, wherein the first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer.

In some embodiments, the first and/or the second amyloid-reactive peptide comprises an amino acid sequence having at least 85% sequence identity to any one of the amino acid sequences set forth as SEQ ID NOs: 1-13.

In some embodiments, the first and/or second human Fc domain is a human IgG1, IgG2, or IgG4 Fc.

In some embodiments, the first and/or second human Fc domain is a human IgG1 Fc.

In some embodiments, the first and/or second human Fc domain comprises an amino acid sequence set forth in SEQ ID NO: 18.

In some embodiments, the first and/or second amyloid-reactive peptide is linked to the first and/or second human Fc domain via a spacer.

In some embodiments, the spacer is a peptide spacer.

In some embodiments, the spacer comprises an amino acid sequence set forth in any one of SEQ ID NOs: 14-17.

In some embodiments, the first polypeptide comprises, from N- to C-terminus, a first human Fc domain, a first spacer, and a first amyloid-reactive peptide, and the second polypeptide comprises, from N- to C-terminus a second human Fc domain, a second spacer, and a second amyloid-reactive peptide.

In some embodiments, the first polypeptide comprises an amino acid sequence set forth in SEQ ID NO: 20, and the second polypeptide comprises an amino acid sequence set forth in SEQ ID NO: 20.

In some embodiments, the amyloid-reactive peptide-Fc fusion protein binds to rVλ6Wil, Aβ, Aβ(1-40), IAAP, ALκ4, ALλ1, or ATTR amyloid.

In some embodiments, in the amyloid-reactive peptide-Fc fusion protein is conjugated to a detectable label.

In another aspect, provided herein is a pharmaceutical composition comprising the amyloid-reactive peptide-Fc fusion protein of any one of paragraphs [0006]-[0017].

In another aspect, provided herein are nucleic acid(s) encoding the amyloid-reactive peptide-Fc fusion protein of any one of paragraphs [0006]-[0017].

In another aspect, provided herein is a vector comprising the nucleic acid(s) of paragraph [0019].

In another aspect, provided herein is a host cell comprising the vector of paragraph [0020].

In some embodiments, the host cell is a mammalian cell, optionally a Chinese hamster ovary (CHO) cell.

In another aspect, provided herein is a method of making an amyloid-reactive peptide-Fc fusion protein comprising culturing the host cell of paragraph [0021] or paragraph [0022] under conditions suitable for expression of the vector encoding the fusion protein.

In some embodiments, the method further comprises recovering the amyloid-reactive peptide-Fc fusion protein.

In another aspect, provided herein is a method of treating an amyloid disease comprising administering a therapeutically effective amount of the amyloid-reactive peptide-Fc fusion protein of any one of paragraphs [0006]-[0017] to an individual in need thereof.

In some embodiments, the amyloid related disease is systemic or localized amyloidosis.

In some embodiments, the amyloid related disease is selected from the group consisting of AL, AH, Aβ2M, ATTR, transthyretin, AA, AApoAI, AApoAII, AGel, ALys, ALEct2, AFib, ACys, ACal, AMed, AIAPP, APro, AIns, APrP, or Aβ amyloidosis.

In some embodiments, treatment with the amyloid-reactive peptide-Fc fusion protein results in the clearance of amyloid.

In another aspect, provided herein is a method of targeting an amyloid deposit for clearance, comprising contacting an amyloid deposit with the amyloid-reactive peptide-Fc fusion protein of any one of paragraphs [0006]-[0017].

In some embodiments, targeting the amyloid deposit for clearance results in clearance of the amyloid deposit.

In some embodiments, clearance results from opsonization of the amyloid deposit.

In some embodiments, the individual is a human.

In another aspect, provided herein is a method of treating an individual suffering from, or suspected to be suffering from, an amyloid-based disease, comprising: determining whether the individual has an amyloid deposit by: detectably labeling the amyloid-reactive peptide-Fc fusion protein of any one of paragraphs [0006]-[0017], administering the labeled amyloid-reactive peptide-Fc fusion protein to the individual, determining whether a signal associated with the detectable label can be detected from the individual; and, if the signal is detected, administering to the individual an amyloidosis treatment.

In some embodiments, if a signal is not detected, monitoring the individual for a later development of an amyloid deposit.

In some embodiments, the method further comprises determining the intensity of the signal and comparing the signal to a threshold value, above which the individual is determined to possess an amyloid deposit.

In some embodiments, the amyloidosis treatment comprises administering the amyloid-reactive peptide-Fc fusion protein of any one of paragraphs [0006]-[0017] to the individual.

In another aspect, provided herein is a method of identifying an amyloid deposit in an individual, comprising detectably labeling the amyloid-reactive peptide-Fc fusion protein of any one of paragraphs [0006]-[0017], administering the amyloid-reactive peptide-Fc fusion protein to the individual, and detecting a signal from the fusion protein.

In some embodiments, the individual is determined to be amyloid free or suffering from monoclonal gammopathy of unknown significance (MGUS), multiple myeloma (MM), or one or more related plasma cell diseases.

### BRIEF DESCRIPTION OF THE DRAWINGS

FIG. 1 shows schematic diagrams of exemplary peptide-Fc constructs along with the nomenclature used herein to refer to each construct.

FIG. 2 shows results of a sodium dodecyl sulfate polyacrylamide gel electrophoresis (SDS-PAGE) analysis of proteins produced in Chinese hamster ovary (CHO) cells with 2% FBS. The lanes show, from left to right, a molecular weight standard, an IgG1 antibody control (“VH9/VL4 IgG1”), a peptide-antibody fusion protein with the peptide p5R fused to the N-terminus of a human IgG (“hFcNV1”), an IgG1 Fc control (“hFc1”) and a peptide-Fc fusion with the peptide p5R fused to the C-terminus of an IgG1 Fc (“hFc1CV1”). The lines indicate the electrophoretic mobility of a native immunoglobulin light chain (lane 2) and control Fc domain (lane 4) to allow comparison with the modified, peptide-fusion light chain (lane 3) and peptide-Fc fusion (lanes 5 and 6). The asterisk on lane 6 indicates an Fc-peptide variant with fully intact peptide associated with the Fc-domain.

FIG. 3 shows the results of a size exclusion chromatography (SEC) analysis of Fcp5RCV1 (light gray line, bottom diagram) and Fcp5RNV1 (black line, top diagram) peptide-Fc fusion proteins. The x-axis shows the time in minutes, the left y-axis shows the absorbance at 280 nm for Fcp5RCV1, and the right y-axis shows the absorbance at 280 nm for Fcp5RNV1.

FIG. 4 shows the results of radioiodinating the Fcp5R CV1 peptide-Fc construct, in comparison to the antibody 11-1F4. The proteins were produced in CHO cells with 2% FBS. Fcp5RCV1 and 11-1F4 are each shown reduced (“Red.”) and not reduced (“NR”). The positions of the 11-1F4 IgG, IgG heavy chain (“HC”), IgG light chain (“LC”), and Fcp5R CV1 are indicated.

FIG. 5 shows the biodistribution of ¹²⁵I-hFc1CV1 (¹²⁵I-I-CV1) in AA mice after 1 hour post injection with ¹²⁵I-hFc1CV1 (black bars), 4 hours post injection (medium gray bars), or 24 hours post injection (light gray bars). The x-axis indicates the tissue measured (including, from left to right, muscle, liver, pancreas, spleen, left kidney, right kidney, stomach, upper intestine, lower intestine, heart, lung, and blood), and the y-axis indicates the level of biodistribution as a percentage of injected dose per gram tissue (mean+SD from three mice per group).

FIG. 6 shows single photon emission computed tomography (SPECT) imaging of ¹²⁵I-hFc1CV1 in AA mice after 1, 4, or 24 hours post injection with ¹²⁵I-hFc1CV1.

FIG. 7 shows microautoradiography (ARG; bottom row) and Congo red staining (top row) of spleen (left), heart (center), and liver (right) tissues showing ¹²⁵I-hFc1CV1 in AA mice 1 hour post injection with ¹²⁵I-hFc1CV1.

FIG. 8 shows microautoradiography (ARG; bottom row) and Congo red staining (top row) of spleen (left), heart (center), and liver (right) tissues showing ¹²⁵I-hFc1CV1 in AA mice 24 hour post injection with ¹²⁵I-hFc1CV1.

FIG. 9 shows pHrodo red-labeled rVλ6Wil fibril uptake by human PMA-activated THP-1 macrophages alone (control), or in the presence of a human (h) Fc1, 1 μg Fc1NV1, 3 μg Fc1NV1, 10 μg Fc1NV1, 1 μg Fc1CV1, 3 μg Fc1CV1, or 10 μg Fc1CV1 for one hour, as indicated from left to right on the x-axis. The y-axis shows the level of rVλ6Wil fibril uptake (measured in fluorescent units). The data represents the mean+standard deviation (n=4).

FIG. 10 shows pHrodo red-labeled rVλ6Wil fibril uptake by human PMA-activated THP-1 macrophages alone (control), or in the presence of 1 μg human Fc1 (hFc1 control), 1 μg hFc1CV1, 3 μg hFc1, 3 μg hFc1CV1, 10 μg hFc1, 10 μg hFc1CV1, 30 μg hFc1, or 30 μg Fcp5R CV1, as indicated from left to right on the x-axis. The y-axis shows the level of rVλ6Wil fibril uptake (measured in fluorescent units), and the error bars represent the standard deviation. hFc1 and Fcp5R CVI were produced by CHO cells. The data represents the mean+standard deviation (n=4).

FIG. 11 shows the binding of hFc1CV1 to rVλ6Wil fibrils (light gray), compared to a human (h) Fc1 control (dark gray). The x-axis shows the concentration of Fcp5R CV1 or hFc1 in nM, and the y-axis shows the amount of bound reagent in fluorescence arbitrary units (au). The EC₅₀ of hFc1CV1 binding to rVλ6Wil fibrils was 2.5 nM.

FIG. 12A-12B shows binding of hFc1CV1 in mice with system amyloid protein A associated amyloidosis. SPECT/Ct images were obtained by detecting radiolabeled Fcp5RCV1 at 1, 4, 24, and 48 hours post injection with Fcp5RCV1. FIG. 12A shows the distribution in various organs at each time point in the AA mice. FIG. 12B shows the distribution in various organs in AA Mice compared to wild-type mice at 48 hours post injection.

FIG. 13A-13B show co-localization of I-125 labeled hFc1CV1 (¹²⁵I Fcp5RCV1) and amyloid in various tissues. ARG (audioradiograph) shows the localization of labeled hFc1CV1 (and CR (congo red) shows localization of amyloid at 1 hour (FIG. 13A) and 24 hours (FIG. 13B) post injection with hFc1CV1.

FIG. 14A-D show the results of an ex vivo phagocytosis assay performed with Fcp5RCV1 or human IgG1 control. Phagocytosis is detected by labeling with the pH sensitive dye succinimidyl-pHrodo red fluorophore.

FIG. 15 shows the results of an ex vivo phagocytosis assay performed with hFc1CV1 on rVλWIL, ALκ, and ALλ fibrils in the presence (+C) or absence of 20% human plasma (a source of complement).

FIG. 16 shows the results of a binding experiment testing the affinity of hFc1CV1 on ATTRV, ATTRwt, rVλWIL, ALκ, and ALλ. ATTRwt is a wild type transthyretin associated amyloidosis. ATTRv is a variant transthyretin associated amyloidosis.

FIG. 17 shows the results of a binding experiment testing the affinity of Fcp5RCV1 on synthetic amyloid-like fibrils Tau 441, α-synuclein, and Aβ(1-40).

FIGS. 18A-18C show immunohistochemical staining to detect hFc1CV1 (top panels) and Congo red fluorescence to detect amyloid fibrils in human tissue sections from individuals with fibril deposits. FIG. 18A shows hFc1CV1 binding to ATTR and ALκ fibrils in human brain tissue sections. FIG. 18B shows hFc1CV1 binding to ALκ, and ALA amyloid deposits in human kidney and liver tissue sections. FIG. 18C shows hFc1CV1 binding to ATTR and ALκ fibrils in human heart tissue sections. Arrows show location of amyloid and binding of hFc1CV1.

### DETAILED DESCRIPTION

Provided herein are amyloid-reactive peptide-Fc fusion proteins which are able to bind to amyloids and induce phagocytosis.

### I. Definitions

As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

Ranges can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect. In certain example embodiments, the term “about” is understood as within a range of normal tolerance in the art, for example within 2 standard deviations of the mean. About can be understood as within 10%, 9%, 8%. 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary.” or “exemplified,” are not meant to show preference, but rather to explain that the aspect discussed thereafter is merely one example of the aspect presented.

The terms amyloids, amyloid deposits, amyloid fibrils, and amyloid fibers refer to insoluble fibrous protein aggregates sharing specific structural traits. The protein aggregates have a tertiary structure, for example, that is formed by aggregation of any of several different proteins and that consists of an ordered arrangement of ß sheets stacked perpendicular to a fiber axis. See Sunde et al., J. Mol. Biol. (1997) 273:729-39. Abnormal accumulation of amyloids in organs may lead to amyloidosis. Although they are diverse in their occurrence, all amyloids have common morphologic properties in that they stain with specific dyes such as Congo red and have a characteristic red-green birefringent appearance in polarized light after staining. Amyloids also share common ultrastructural features and common x-ray diffraction and infrared spectra.

Amyloidosis refers to a pathological condition or disease characterized by the presence of amyloids, such as the presence of amyloid deposits. “Amyloid diseases” or “amyloidosis” are diseases associated with the formation, deposition, accumulation or persistence of amyloid fibrils. Such diseases include, but are not limited to, Alzheimer's disease, Down's syndrome, hereditary cerebral hemorrhage with amyloidosis of the Dutch type, and cerebral beta-amyloid angiopathy. Other amyloid diseases such as systemic AA amyloidosis, AL amyloidosis, ATTR amyloidosis, ALect2 amyloidosis, and IAPP amyloidosis of type II diabetes are also amyloid diseases.

Amyloidogenic refers to producing or tending to produce amyloid deposits. For example, certain soluble monomeric proteins can undergo extensive conformational changes leading to their aggregation into well-ordered, unbranching, 8- to 10-nm wide fibrils, which culminate in the formation of amyloid aggregates. More than thirty proteins, for example, have been found to form amyloid deposits (or amyloids) in man. Not all proteins within the class of diverse proteins, such as immunoglobulin light chains, are capable of forming amyloid, i.e., some proteins are non-amyloidogenic, meaning that they do not tend to form amyloids. Other proteins of the class, however, can form amyloid deposits and are thus amyloidogenic. Furthermore, within the class of light chain protein, some may be deemed more “amyloidogenic” than others based upon the case with which they form amyloid fibrils. Certain light chain proteins are deemed non-amyloidogenic or less amyloidogenic because of their inability to readily form amyloid fibrils in patients or in vitro.

Animal: Living multi-cellular vertebrate organisms, a category that includes, for example, mammals and birds. The term mammal includes both human and non-human mammals. Similarly, the terms “subject” and “individual” includes both human and veterinary individuals. In some examples an animal is an individual suffering from an amyloid disease.

Clearance: The terms “clear” or “clearance” refer to reducing or removing by a measurable degree. For example, the clearance of an amyloid deposit as described herein relates to reducing or removing the deposit to a measurable or discernable degree. Clearance may result in 100% removal, but is not required to. Rather, clearance may result in less than 100% removal, such as about 10%, 20%, 30%, 40%, 50%, 60% or more removal.

Conjugate: As used herein, the term “conjugate” refers to the product of coupling or joining of two or more materials, the resulting product having at least two distinct elements, such as at least two domains. The coupled materials may be the same or may be different. Such a coupling may be via one or more linking groups. A “protein conjugate,” for example, results from the coupling of two or more amino acid sequences. A conjugate of two proteins, for example, results in a single protein that has a domain corresponding to each of the individually joined proteins.

Effective amount or Therapeutically effective amount: The amount of agent that is sufficient to prevent, treat (including prophylaxis), reduce and/or ameliorate the symptoms and/or underlying causes of any of a disorder or disease, for example to prevent, inhibit, and/or amyloidosis. In some embodiments, an “effective amount” is sufficient to reduce or eliminate a symptom of a disease. An effective amount can be administered one or more times.

Inhibit: To reduce by a measurable degree. Inhibition does not, for example, require complete loss of function or complete cessation of the aspect being measured. For example, inhibiting plaque formation can mean stopping further growth of the plaque, slowing further growth of the plaque, or reducing the size of the plaque.

Inhibiting or treating a disease: Inhibiting the full development of a disease or condition, for example, inhibiting amyloidosis. “Treatment” refers to a therapeutic intervention that ameliorates a sign or symptom of a disease or pathological condition after it has begun to develop. The term “ameliorating.” with reference to a disease or pathological condition, refers to any observable beneficial effect of the treatment. The beneficial effect can be evidenced, for example, by a delayed onset of clinical symptoms of the disease in a susceptible individual, a reduction in severity of some or all clinical symptoms of the disease, a slower progression of the disease, an improvement in the overall health or well-being of the individual, or by other parameters well known in the art that are specific to the particular disease. A “prophylactic” treatment is a treatment administered to an individual who does not exhibit signs of a disease or exhibits only early signs for the purpose of decreasing the risk of developing pathology.

With regard to amyloid deposit formation, “inhibition” refers to the prevention of reduction in the formation of the amyloid deposit, such as when compared to a control. For example, inhibition may result in a reduction of about 10%, 20%, 30%, 40%, 50%, 60% or more of an amyloid deposit as compared to a control.

Label refers to any detectable compound or composition that is conjugated directly or indirectly to another molecule to facilitate detection of that molecule. Specific, non-limiting examples of labels include fluorescent tags, chemiluminescent tags, haptens, enzymatic linkages, and radioactive isotopes. A protein that is “detectably-labeled,” for example, means that the presence of the protein can be determined by a label associated with the protein.

Isolated: An “isolated” biological component, such as a peptide (for example one or more of the peptides disclosed herein), cell, nucleic acid, or serum samples has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Join: As used herein, the term “join,” “joined.” “link,” or “linked” refers to any method known in the art for functionally connecting proteins and/or protein domains. For example, one protein domain may be linked to another protein domain via a covalent bond, such as in a recombinant fusion protein, with or without intervening sequences or domains. Joined also includes, for example, the integration of two sequences together, such as placing two nucleic acid sequences together in the same nucleic acid strand so that the sequences are expressed together.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

cDNA refers to a DNA that is complementary or identical to an mRNA, in either single stranded or double stranded form.

Encoding refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Pharmaceutically acceptable carriers: The pharmaceutically acceptable carriers of use are conventional. Remington's Pharmaceutical Sciences, by E. W. Martin, Mack Publishing Co., Easton, PA, 19^{th} Edition (1995), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. In some examples, a peptide is one or more of the peptides disclosed herein.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene 73: 237-244, 1988; Higgins & Sharp CABIOS 5: 151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to an individual or a cell.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses.

A subject or an individual refers to a mammal, for example, a human. The individual may be a human patient. An individual may be a patient suffering from or suspected of suffering from a disease or condition and may be in need of treatment or diagnosis or may be in need of monitoring for the progression of the disease or condition. The patient may also be in on a treatment therapy that needs to be monitored for efficacy. In some example embodiments, an individual includes an individual suffering from amyloidosis, such as Alzheimer's, Huntington's or prion diseases, or peripheral amyloidosis such as seen in patients with light chain (AL) amyloidosis and type 2 diabetes.

The terms treating or treatment refer to a therapeutic intervention that ameliorates a sign or symptom of a disease or pathological condition after it has begun to develop. The term “ameliorating.” with reference to a disease or pathological condition, refers to any observable beneficial effect of the treatment. The beneficial effect can be evidenced, for example, by a delayed onset of clinical symptoms of the disease in a susceptible individual, a reduction in severity of some or all clinical symptoms of the disease, a slower progression of the disease, an improvement in the overall health or well-being of the individual, or by other parameters well known in the art that are specific to the particular disease. A “prophylactic” treatment is a treatment administered to an individual who does not exhibit signs of a disease or exhibits only carly signs for the purpose of decreasing the risk of developing pathology.

### II. Amyloid-Reactive Peptide-Fc Fusion Proteins

Provided herein are amyloid-reactive peptide-Fc fusion proteins. In some embodiments, the amyloid-reactive peptide-Fc fusion protein comprises a first polypeptide and a second polypeptide, wherein the first polypeptide comprises a first amyloid-reactive peptide linked to a first human Fc domain, wherein the second polypeptide comprises a second amyloid-reactive peptide linked to a second human Fc domain, and wherein the first and the second human Fc domains form a dimer. In some embodiments, the amyloid-reactive peptide-Fc fusion protein is a homodimer. The amyloid-reactive peptide-Fc fusion proteins can be used to treat a subject suffering from amyloidosis, for example, such as by administering an amyloid-reactive peptide-Fc fusion protein of the present disclosure to an individual.

In some embodiment, the first and/or the second amyloid-reactive peptide comprises an amino acid sequence as shown in Table 1, below. In some embodiments, one or more of the peptides shown in Table 1 below can be joined to a human Fc region through the N-terminus of the human Fc region or the C-terminus of the human Fc region, thereby forming an amyloid-reactive peptide-Fc fusion protein. In some embodiments, the first and/or the second amyloid-reactive peptide comprises two or more of the peptides shown in Table 1, which can be joined to a single human Fc region. For example, two of the amyloid reactive peptides can be joined with a single human Fc region.

TABLE 1
Exemplary Amyloid-Reactive Peptide Sequences

| Peptide | PRIMARY SEQUENCE: | SEQ ID NO |
|---|---|---|
| p5 | KAQKA QAKQA KQAQK | SEQ ID NO: 1 |
| | AQKAQ AKQAK Q | |
| p5R | RAQRA QARQA RQAQR | SEQ ID NO: 2 |
| | AQRAQ ARQAR Q | |
| p8 | KAKAKAKAKA KAKAK | SEQ ID NO: 3 |
| p9 | KAQAK AQAKA QAKAQ | SEQ ID NO: 4 |
| | AKAQA KAQAK AQAK | |
| p19 | KAQQA QAKQA QQAQK | SEQ ID NO: 5 |
| | AQQAQ AKQAQ Q | |
| p20 | QAQKA QAQQA KQAQQ | SEQ ID NO: 6 |
| | AQKAQ AQQAK Q | |
| p31 | KAQKA QAKQA KQAQK | SEQ ID NO: 7 |
| | AQKAQ AKQAK Q | |
| p37 | KTVKT VTKVT KVTVK | SEQ ID NO: 8 |
| | TVKTV TKVTK V | |
| p42 | VYKVK TKVKT KVKTK | SEQ ID NO: 9 |
| | VKT | |
| p43 | AQAYS KAQKA QAKQA | SEQ ID NO: 10 |
| | KQAQK AQKAQ AKAK | |
| | Q | |
| p44 | AQAYA RAQRA QARQA | SEQ ID NO: 11 |
| | RQAQR AQRAQ ARQAR | |
| | Q | |
| p5+14 | KAQKA QAKQA KQAQK | SEQ ID NO: 12 |
| | AQKAQ AKQAK QAQKA | |
| | QKAQA KQAKQ | |
| p5R+14 | RAQRA QARQA RQAQR | SEQ ID NO: 13 |
| | AQRAQ ARQAR QAQRA | |
| | QRAQA RQARQ | |

Without wishing to be bound by any particular theory, it is believed that the peptide domain of the amyloid-reactive peptide-Fc fusion protein, when administered to an individual, targets the amyloid-reactive peptide-Fc fusion protein to the amyloid deposits. The Fc domain then triggers an immune response at the site of the amyloid, thereby resulting in removal of the amyloid, such as by opsonization. In addition, the amyloid-reactive peptide-Fc fusion protein is believed to have a longer half-life than the amyloid-reactive peptides alone. In certain example embodiments, contacting an amyloid deposit with a fusion protein of the present disclosure results in a half-life that is increased by about 10%, 20%, 30%, 40%, 50%, 60%, 70%, 80% or more as compared to contacting an amyloid deposit with the amyloid-reactive peptide alone. As such, the amyloid-reactive peptide-Fc fusion protein, when administered to an individual, can exert its immunostimulatory effects longer at the site of the amyloid deposit, thereby increasing the immune response at the site of the amyloid deposit.

In some embodiments, the amyloid-reactive peptides of the amyloid-reactive peptide-Fc fusion proteins described herein include an amino acid sequence that is at least 80%, 85%, 90% or more identical to the amino acid sequence set forth as any one of SEQ ID NOS: 1-13, such as at least 95%, at least 96%, at least 97%, at least 98%, at least 99%, or 100% identical to the amino acid sequence set forth as any one of SEQ ID NOS: 1-13. In some embodiments, the amyloid-reactive peptides linked to the human Fc region may comprise or consist of from about 10 to about 55 amino acids. The amyloid-reactive peptides of the present invention may, for example, comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in international patent application WO2016032949, which is hereby incorporated herein in its entirety. In some embodiments, the first and/or the second amyloid-reactive peptide comprises an amino acid sequence having at least 80%, 85%, 90%, 95% or more sequence identity to any one of the amino acid sequences set forth as SEQ ID NOs: 1-13. In some embodiments, the first and the second amyloid-reactive peptide comprises an amino acid sequence having at least 80%, 85%, 90%, 95% or more sequence identity to any one of the amino acid sequences set forth as SEQ ID NOs: 1-13. In some embodiments, the first and/or the second amyloid-reactive peptide comprises the amino acid sequences set forth as SEQ ID NOs: 1-13 comprising 1, 2, 3, 4, or 5 amino acid substitutions. In some embodiments, the first and the second amyloid-reactive peptide comprises an amino acid sequence set forth in SEQ ID NO: 1. In some embodiments, the first and the second amyloid-reactive peptide comprises an amino acid sequence set forth in SEQ ID NO: 2. In some embodiments, the first and the second amyloid-reactive peptide comprises an amino acid sequence set forth in SEQ ID NO: 12. In some embodiments, the first and the second amyloid-reactive peptide comprises an amino acid sequence set forth in SEQ ID NO: 13.

The amino acids forming all or a part of the amyloid-reactive peptides linked to the human Fc regions may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids.

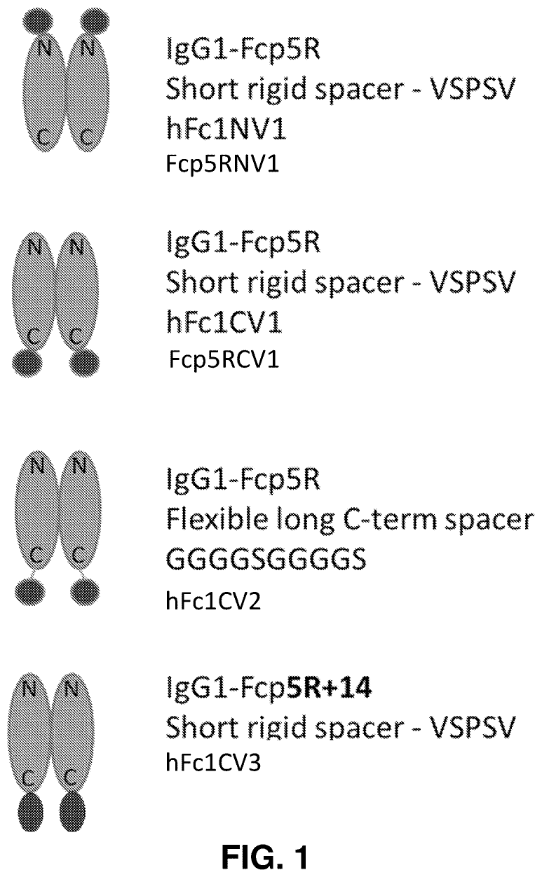

In some embodiments, the first amyloid-reactive peptide is linked to the N-terminus of the first human Fc domain, and the second amyloid-reactive peptide is linked to the N-terminus of the second human Fc domain. In some embodiments, the first amyloid-reactive peptide is linked to the C-terminus of the first human Fc domain, and the second amyloid-reactive peptide is linked to the C-terminus of the second human Fc domain. Exemplary structures of amyloid-reactive peptide-Fc fusion proteins are provided in FIG. 1.

In some embodiments, the first and/or second human Fc domain is a human IgG1, IgG2, or IgG4 Fc. In some embodiments, the first and/or second human Fc domain is a human IgG1 Fc. In some embodiments, the first and second human Fc domain is a human IgG1 Fc. In some embodiments, the first and/or second human Fc domain comprises the amino acid sequence set forth in SEQ ID NO: 18. In some embodiments, the first and second human Fc domain comprises the amino acid sequence set forth in SEQ ID NO: 18. The amino acid sequence of SEQ ID NO: 18 is provided below.

Human IgG1 Fc
(SEQ ID NO: 18)
DKTHTCPPCPAPELLGGPSVFLFPPKPKDTL
MISRTPEVTCVVVDVSHEDPEVKFNWYVDGV
EVHNAKTKPREEQYNSTYRVVSVLIVLHQDW
LNGKEYKCKVSNKALPAPIEKTISKAKGQPR
EPQVYTLPPSRDELTKNQVSLTCLVKGFYPS
DIAVEWESNGQPENNYKTTPPVLDSDGSFFL
YSKLTVDKSRWQQGNVFSCSVMHEALHNHYT
QKSLSLSPGK

In some embodiments, the first and/or second human Fc domain is an Fc variant with enhanced effector function(s). In some embodiments, the first and/or second human Fc domain is an Fc variant with an enhanced ability to promote phagocytosis. In some embodiments, the first and/or second human Fc domain is an Fc variant with an enhanced ability to bind to an FcγR. In some embodiments, the first and/or second human Fc domain is an Fc variant with enhanced ability to recruit complement. In some embodiments, the first and/or second human Fc domain comprises one or more amino acid substitutions that confer enhanced effector function and/or enhanced binding to an FcγR. Such amino acid substitutions have been described, for example, in International Publication Nos. WO2004/099249, WO2005/063815, WO2006/019447, WO2006/020114, WO2007/041635, WO2009/058492, WO2009/086320, and U.S. Publication Nos. US20070224192 and US20080161541, each of which are hereby incorporated by reference. In some embodiments, the human Fc domain with one or more amino acid substitutions has an enhanced ability to promote phagocytosis. In some embodiments, the first and/or second human Fc domain is glycoengineered. In some embodiments, the glycoengineered human Fc domain has an enhanced ability to promote phagocytosis.

In some embodiments, the amyloid-reactive peptide-Fc fusion protein comprises a spacer sequence of amino acids between the human Fc region and the amyloid-reactive peptide. In some embodiments, the first and/or second amyloid-reactive peptide is linked to the first and/or second human Fc domain via a spacer. In some embodiments, the spacer is a peptide spacer. In some embodiments, the first and/or second amyloid-reactive peptide is fused to the first and/or second human Fc domain via a peptide spacer. In some embodiments, the spacer is a flexible spacer peptide. In some embodiments, the spacer comprises glycine and serine residues. In some embodiments, the spacer comprises a GGGGS motif. In some embodiments, the spacer consists of glycine and serine residues. In some embodiments, the spacer is a rigid spacer peptide. In some embodiments, the spacer is uncharged. In some embodiments, the spacer is a glycine serine linker. In some embodiments, the spacer comprises a glycine serine linker. In some embodiments the spacer comprises or consist of from about 3 to about 55 amino acids. The spacer peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer peptide is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the spacer peptide comprises 15 amino acids. In some embodiments, the spacer peptide comprises an amino acid sequence as set forth in Table 2, below. In some embodiments, the spacer comprises an amino acid sequence set forth in SEQ ID NOs: 14-17.

TABLE 2
Exemplary Spacer Sequences

| | Amino Acid | |
|---|---|---|
| Description | Sequence | SEQ ID NO |
| Short, rigid | VSPSV | SEQ ID |
| spacer | | NO: 14 |
| Long, rigid | VSPSV | SEQ ID |
| spacer | VSPSV | NO: 15 |
| Flexible, short | GGSGG | SEQ ID |
| spacer | | NO: 16 |
| Flexible, long | GGGGS | SEQ ID |
| spacer | GGGGS | NO: 17 |

In some embodiments, the first polypeptide comprises, from N- to C-terminus, a first amyloid-reactive peptide, a first spacer, and a first human Fc domain, and the second polypeptide comprises, from N- to C-terminus, a second amyloid-reactive peptide, a second spacer, and a second human Fc domain. In some embodiments, the first and second polypeptides have the same sequence. In some embodiments, the first and second polypeptides have different sequences. In some embodiments, the first polypeptide comprises, from N- to C-terminus, a first amyloid-reactive peptide set forth in SEQ ID NO: 2, a first spacer, and a first human Fc domain, and the second polypeptide comprises, from N- to C-terminus, a second amyloid-reactive peptide set forth in SEQ ID NO: 2, a second spacer, and a second human Fc domain. In some embodiments, the first polypeptide comprises, from N- to C-terminus, a first amyloid-reactive peptide, a first spacer, and a first human IgG1 Fc domain, and the second polypeptide comprises, from N- to C-terminus, a second amyloid-reactive peptide, a second spacer, and a second human IgG1 Fc domain. In some embodiments, the first polypeptide comprises, from N- to C-terminus, a first amyloid-reactive peptide, a first short, rigid spacer, and a first human Fc domain, and the second polypeptide comprises, from N- to C-terminus, a second amyloid-reactive peptide, a second short, rigid spacer, and a second human Fc domain. In some embodiments, the short rigid spacer comprises the sequence set forth in SEQ ID NO:14. In some embodiments, the first polypeptide comprises, from N- to C-terminus, a first amyloid-reactive peptide set forth in SEQ ID NO: 2, a first short, rigid spacer, and a first human IgG1 Fc domain, and the second polypeptide comprises, from N- to C-terminus, a second amyloid-reactive peptide set forth in SEQ ID NO: 2, a second short, rigid spacer, and a second human IgG1 Fc domain. In some embodiments, the first polypeptide comprises an amino acid sequence sharing at least 70%, 75%, 80%, 85%, 90%, 95%, 96%, 97%, 98%, or 99% identity with the amino acid sequence set forth in SEQ ID NO: 19, and the second polypeptide comprises an amino acid sequence sharing at least 70%, 75%, 80%, 85%, 90%, 95%, 96%, 97%, 98%, or 99% identity with the amino acid sequence set forth in SEQ ID NO: 19. In some embodiments, the first polypeptide comprises an amino acid sequence set forth in SEQ ID NO: 19, and the second polypeptide comprises an amino acid sequence set forth in SEQ ID NO: 19. In some embodiments, the amyloid-reactive peptide-Fc fusion protein comprises the structure and/or amino acid sequence of hFc1NV1.

In some embodiments, the first polypeptide comprises, from N- to C-terminus, a first human Fc domain, a first spacer, and a first amyloid-reactive peptide, and the second polypeptide comprises, from N- to C-terminus a second human Fc domain, a second spacer, and a second amyloid-reactive peptide. In some embodiments, the first polypeptide comprises, from N- to C-terminus, a first human Fc domain, a first spacer, and a first amyloid-reactive peptide set forth in SEQ ID NO: 2, and the second polypeptide comprises, from N- to C-terminus a second human Fc domain, a second spacer, and a second amyloid-reactive peptide set forth in SEQ ID NO: 2. In some embodiments, the first polypeptide comprises, from N- to C-terminus, a first human IgG1 Fc domain, a first spacer, and a first amyloid-reactive peptide, and the second polypeptide comprises, from N- to C-terminus a second human IgG1 Fc domain, a second spacer, and a second amyloid-reactive peptide. In some embodiments, the first polypeptide comprises, from N- to C-terminus, a first human Fc domain, a first short, rigid spacer, and a first amyloid-reactive peptide, and the second polypeptide comprises, from N- to C-terminus a second human Fc domain, a second short, rigid spacer, and a second amyloid-reactive peptide. In some embodiments, the first polypeptide comprises, from N- to C-terminus, a first human IgG1 Fc domain, a first short, rigid spacer, and a first amyloid-reactive peptide set forth in SEQ ID NO: 2, and the second polypeptide comprises, from N- to C-terminus a second human IgG1 Fc domain, a second short, rigid spacer, and a second amyloid-reactive peptide set forth in SEQ ID NO: 2. In some embodiments, the short rigid spacer comprises the amino acid sequence set forth in SEQ ID NO: 14. In some embodiments, the first polypeptide comprises an amino acid sequence sharing at least 70%, 75%, 80%, 85%, 90%, 95%, 96%, 97%, 98%, or 99% identity with the amino acid sequence set forth in SEQ ID NO: 20, and the second polypeptide comprises an amino acid sequence sharing at least 70%, 75%, 80%, 85%, 90%, 95%, 96%, 97%, 98%, or 99% identity with the amino acid sequence set forth in SEQ ID NO: 20. In some embodiments, the first polypeptide comprises an amino acid sequence set forth in SEQ ID NO: 20, and the second polypeptide comprises an amino acid sequence set forth in SEQ ID NO: 20. In some embodiments, the amyloid-reactive peptide-Fc fusion protein comprises the structure and/or amino acid sequence of hFc1CV1.

In some embodiments, the first polypeptide comprises, from N- to C-terminus, a first human Fc domain, a first flexible, long spacer, and a first amyloid-reactive peptide, and the second polypeptide comprises, from N- to C-terminus a second human Fc domain, a second flexible, long spacer, and a second amyloid-reactive peptide. In some embodiments, the first polypeptide comprises, from N- to C-terminus, a first human Fc domain, a first flexible, long spacer, and a first amyloid-reactive peptide set forth in SEQ ID NO: 2, and the second polypeptide comprises, from N- to C-terminus a second human Fc domain, a second flexible, long spacer, and a second amyloid-reactive peptide set forth in SEQ ID NO: 2. In some embodiments, the first polypeptide comprises, from N- to C-terminus, a first human IgG1 Fc domain, a first flexible, long spacer, and a first amyloid-reactive peptide set forth in SEQ ID NO: 2, and the second polypeptide comprises, from N- to C-terminus a second human IgG1 Fc domain, a second flexible, long spacer, and a second amyloid-reactive peptide set forth in SEQ ID NO: 2. In some embodiments, the first polypeptide comprises, from N- to C-terminus, a first human IgG1 Fc domain, a first spacer, and a first amyloid-reactive peptide set forth in SEQ ID NO: 13, and the second polypeptide comprises, from N- to C-terminus a second human IgG1 Fc domain, a second spacer, and a second amyloid-reactive peptide set forth in SEQ ID NO: 13. In some embodiments, the first polypeptide comprises, from N- to C-terminus, a first human Fc domain, a first spacer, and a first amyloid-reactive peptide set forth in SEQ ID NO: 13, and the second polypeptide comprises, from N- to C-terminus a second human Fc domain, a second spacer, and a second amyloid-reactive peptide set forth in SEQ ID NO: 13. In some embodiments, the first polypeptide comprises, from N- to C-terminus, a first human Fc domain, a first flexible, long spacer, and a first amyloid-reactive peptide set forth in SEQ ID NO: 13, and the second polypeptide comprises, from N- to C-terminus a second human Fc domain, a second flexible, long spacer, and a second amyloid-reactive peptide set forth in SEQ ID NO: 13. In some embodiments, the first polypeptide comprises, from N- to C-terminus, a first human IgG1 Fc domain, a first flexible, long spacer, and a first amyloid-reactive peptide set forth in SEQ ID NO: 13, and the second polypeptide comprises, from N- to C-terminus a second human IgG1 Fc domain, a second flexible, long spacer, and a second amyloid-reactive peptide set forth in SEQ ID NO: 13. In some embodiments, the spacer comprises the amino acid sequence of SEQ ID NO:17.

In some embodiments, the first polypeptide comprises an amino acid sequence sharing at least 70%, 75%, 80%, 85%, 90%, 95%, 96%, 97%, 98%, or 99% identity with the amino acid sequence set forth in SEQ ID NO: 21, and the second polypeptide comprises an amino acid sequence sharing at least 70%, 75%, 80%, 85%, 90%, 95%, 96%, 97%, 98%, or 99% identity with the amino acid sequence set forth in SEQ ID NO: 21. In some embodiments, the first polypeptide comprises an amino acid sequence set forth in SEQ ID NO: 21, and the second polypeptide comprises an amino acid sequence set forth in SEQ ID NO: 21.

In some embodiments, the first polypeptide comprises an amino acid sequence sharing at least 70%, 75%, 80%, 85%, 90%, 95%, 96%, 97%, 98%, or 99% identity with the amino acid sequence set forth in SEQ ID NO: 22, and the second polypeptide comprises an amino acid sequence sharing at least 70%, 75%, 80%, 85%, 90%, 95%, 96%, 97%, 98%, or 99% identity with the amino acid sequence set forth in SEQ ID NO: 22. In some embodiments, the first polypeptide comprises an amino acid sequence set forth in SEQ ID NO: 22, and the second polypeptide comprises an amino acid sequence set forth in SEQ ID NO: 22.

In some embodiments, provided herein are fusion proteins comprising a first polypeptide and a second polypeptide, wherein the first polypeptide comprises a first amyloid-reactive peptide and a second amyloid-reactive peptide linked to a first human Fc domain, wherein the second polypeptide comprises a third amyloid-reactive peptide and a fourth amyloid-reactive peptide linked to a second human Fc domain, and wherein the first and the second human Fc domains form a dimer. In some embodiments, the first and second human Fc domains form a dimer by covalent linkage in an antibody hinge region. In some embodiments, the first and second human Fc domains are linked by a disulfide bond. In some embodiments, the amyloid-reactive peptide-Fc fusion protein is a homodimer. In some embodiments, the first polypeptide comprises, from N- to C-terminus, a first amyloid-reactive peptide, a first spacer, a first human Fc domain, a second spacer, and a second amyloid-reactive peptide, and the second polypeptide comprises, from N- to C-terminus, a third amyloid-reactive peptide, a third spacer, a second human Fc domain, a fourth spacer, and a fourth amyloid-reactive peptide. In some embodiments, the first, second, third, and fourth amyloid-reactive peptide is any one of the amyloid-reactive peptides described herein, e.g., any one of the amyloid-reactive peptides described in Table 1. In some embodiments, the first, second, third, and fourth is any one of the spacers described herein.

In some embodiments, the amyloid-reactive peptide-Fc fusion proteins described herein bind to amyloid deposits or fibrils. In some embodiments, the amyloid-reactive peptide-Fc fusion protein binds to one or more amyloidogenic peptides in amyloids. In some embodiments, the amyloid-reactive peptide-Fc fusion protein binds to heparin sulfate glycosoaminoglycans in the amyloid. In some embodiments, the fusion proteins bind to human fibrils. In some embodiments, the fusion proteins bind to synthetic fibrils. In some embodiments, the amyloid-reactive peptide-Fc fusion protein binds to rVλ6Wil fibrils, Per125 wtATTR extract, KEN hATTR extract, SHI ALλ liver extract, and/or TAL ALκ liver extract. In some embodiments, amyloids bound by the amyloid-reactive peptide-Fc fusion protein comprise an amyloidogenic λ6 variable domain protein (Vλ6Wil) or an amyloidogenic immunoglobulin light chain (AL), Aβ(1-40) amyloid-like fibril or an amyloidogenic AB precursor protein, or serum amyloid protein A (AA). In some embodiments, the amyloid-reactive peptide-Fc fusion protein binds to rVλ6Wil, Aβ, Aβ(1-40), IAAP, ALK, ALA, or ATTR amyloid. In some embodiments, the amyloid-reactive peptide-Fc fusion protein binds to ALκ4, ALλ amyloid. 1 In other embodiments, the amyloids bound by the humanized antibody or the antibody-peptide fusion protein comprise amyloidogenic forms of immunoglobulin heavy chain (AH), β₂-microglobulin (Aβ₂M), transthyretin variants (ATTR), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, Alλ1 other amyloidogenic peptides. The amyloidogenic peptides bound by the humanized antibody or the antibody-peptide fusion protein can be a protein, a protein fragment, or a protein domain. In some embodiments, the amyloid deposits or amyloid fibrils comprise recombinant amyloidogenic proteins. In some embodiments, the amyloids are part of the pathology of a disease.

In some embodiments, the amyloid-reactive peptide Fc-fusion protein has pan-amyloid reactivity and is able to bind to diverse amyloid types in various amyloid tissues. In some embodiments, the amyloid-reactive peptide-Fc fusion protein is able to bind to amyloid in the central nervous system. In some embodiments, the amyloid-reactive peptide-Fc fusion protein is able to bind to amyloid in the brain. In some embodiments, the amyloid-reactive peptide-Fc fusion protein is able to bind to tau fibrils and/or alpha synuclein aggregates.

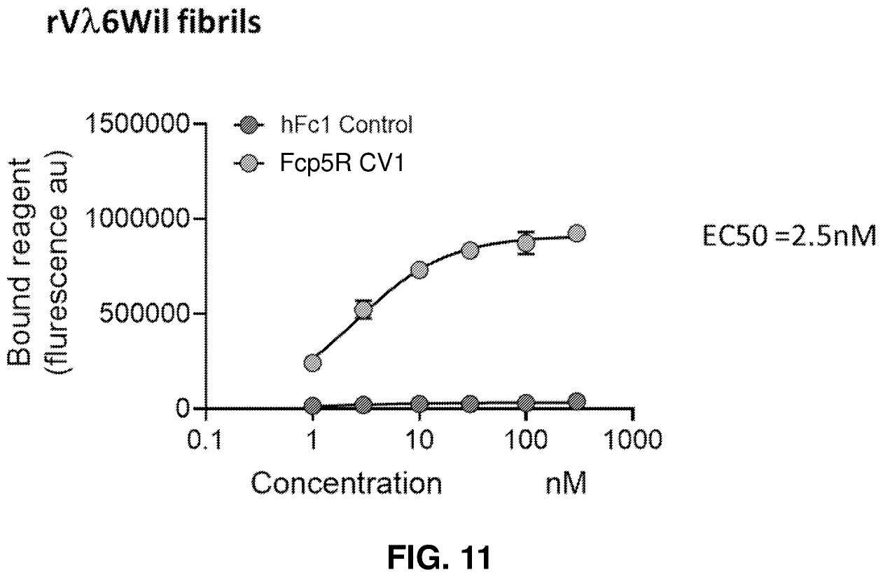

In some embodiments, the amyloid-reactive peptide-Fc fusion protein binds to human amyloid fibrils with a half maximal effective concentration (EC₅₀) that is less than about 1, 10, 100, or 1000 nM. In some embodiments, the amyloid-reactive peptide-Fc fusion protein binds to human amyloid fibrils with an EC₅₀ that is about 1, 2, 3, 4, 5, 6, 7, 8, 9, 10, 15, 20, 25, 30, 35, 40, 45, 50, 55, 60, 65, 70, 75, 80, 85, 90, 95, 100, 100, 250, 500, 750, or 1000 nM, including any value or range between these values. In some embodiments, the amyloid-reactive peptide-Fc fusion protein binds to human amyloid fibrils with an EC₅₀ that is about 1 nM, 2 nM, 2.5 nM, 3 nM, 4 nM, or 5 nM. In some embodiments, the amyloid-reactive peptide-Fc fusion protein binds to human amyloid fibrils with an EC₅₀ that is about 2.5 nM. In some embodiments, the amyloid-reactive peptide-Fc fusion protein binds to human amyloid fibrils with an EC₅₀ that is less than about 3 nM, 4 nM, 5 nM, 10 nM, 20 nM, 80 nM, or 100 nM. In some embodiments, the amyloid-reactive peptide-Fc fusion protein binds to human amyloid fibrils with an EC₅₀ that is less than the EC₅₀ of a control human Fc region binding to human amyloid fibrils. In some embodiments, the amyloid-reactive peptide-Fc fusion protein binds to amyloid deposits or fibrils to a greater extent than a human Fc1 control. Methods for calculating EC₅₀s are known in the art, and include, for example, surface plasmon resonance. In some embodiments, the EC₅₀ is determined by measuring binding to rVλ6Wil fibrils. An exemplary method of measuring binding to amyloid fibrils is provided in Example 4, as shown in FIG. 11.

In some embodiments, binding of the amyloid-reactive peptide-Fc fusion protein to human amyloid promotes the phagocytosis of human amyloid fibrils. In some embodiments, the amyloid-reactive peptide-Fc fusion protein opsonizes human amyloid fibrils. In some embodiments, the amyloid-reactive peptide-Fc fusion protein opsonizes rVλ6Wil fibrils. In some embodiments, contacting human amyloid fibrils with a amyloid-reactive peptide-Fc fusion protein of the present disclosure in the presence of macrophages promotes the uptake of the human amyloid fibrils by the macrophages. In some embodiments, contacting human amyloid fibrils with a amyloid-reactive peptide-Fc fusion protein of the present disclosure in the presence of macrophages promotes the opsonization of the human amyloid fibrils. In some embodiments, binding of the amyloid-reactive peptide-Fc fusion protein to human amyloid promotes the phagocytosis of human amyloid fibrils to an equal or greater extent than a control molecule (e.g., a human Fc region). In some embodiments, the amyloid-reactive peptide-Fc fusion protein promotes antibody-dependent cellular phagocytosis.

In some embodiments, the amyloid-reactive peptide-Fc fusion protein is conjugated to a detectable label. In some embodiments, the detectable label is selected from the group consisting of radionuclides (e.g., I-¹²⁴, I-¹²⁵, I-¹²³, I-¹³¹, Zr-⁸⁹, Tc-^{99m}, Cu-⁶⁴, Br-⁷⁶, F-¹⁸); enzymes (horse radish peroxidase); biotin; and fluorophores, etc. Any means known in the art for detectably labeling a protein can be used and/or adapted for use with the methods described herein. For example, the amyloid-reactive peptide-Fc fusion protein can be radiolabeled with a radioisotope, or labeled with a fluorescent tag or a chemiluminescent tag. Example radioisotopes include, for example, ¹⁸F, ¹¹¹In, ^{99m}Tc, and ¹²³I, and ¹²⁵I. These and other radioisotopes can be attached to the amyloid-reactive peptide-Fc fusion protein using well known chemistry that may or not involve the use of a chelating agent, such as DTPA or DOTA covalently linked to the 1 the amyloid-reactive peptide-Fc fusion protein, for example. Example fluorescent or chemiluminescent tags include fluorescein, Texas red, rhodamine, Alexa dyes, and luciferase that can be conjugated to the amyloid-reactive peptide-Fc fusion protein by reaction with lysine, cysteine, glutamic acid, and aspartic acid side chains. In one example embodiment, the label is detected using a fluorescent microplate reader, or fluorimeter, using the excitation and emission wavelengths appropriate for the tag that is used. Radioactive labels can be detected, for example, using a gamma or scintillation counter depending on the type of radioactive emission and by using energy windows suitable for the accurate detection of the specific radionuclide. However, any other suitable technique for detection of radioisotopes can also be used to detect the label. In some embodiments, the detectable label is ¹²⁵I.

Also provided herein are pharmaceutical compositions comprising any of the amyloid-reactive peptide-Fc fusion proteins described herein. In some embodiments, the pharmaceutical composition further comprises a pharmaceutically acceptable carrier.

### III. Nucleic Acids, Vectors, Host Cells, and Methods of Making Fusion Proteins

Also provided herein is nucleic acid encoding a amyloid-reactive peptide-Fc fusion protein. In some embodiments, the nucleic acid encodes any of the amyloid-reactive peptide-Fc fusion proteins described herein.

In some embodiments, the nucleic acid provided herein are in one or more vectors. In some embodiments, the vector comprises the nucleic acid(s) encoding a amyloid-reactive peptide-Fc fusion protein of the present disclosure.

In some embodiments, the amyloid-reactive peptide-Fc fusion protein is a homodimer. In some embodiments, the vector comprises a nucleic acid encoding both the first and the second polypeptide of the amyloid-reactive peptide-Fc fusion protein.

In some embodiments, the amyloid-reactive peptide-Fc fusion protein is a heterodimer. In some embodiments, the vector comprises a first nucleic acid encoding the first polypeptide of the amyloid-reactive peptide-Fc fusion protein, and a second nucleic acid encoding the second polypeptide of the amyloid-reactive peptide-Fc fusion protein. In some embodiments, a first vector comprises a first nucleic acid encoding the first polypeptide of the amyloid-reactive peptide-Fc fusion protein, and a second vector comprises a second nucleic acid encoding the second polypeptide of the amyloid-reactive peptide-Fc fusion protein.

For amyloid-reactive peptide-Fc fusion protein production, the amyloid-reactive peptide-Fc fusion protein expression vector(s) may be introduced into appropriate production cell lines known in the art. Introduction of the expression vector(s) may be accomplished by co-transfection via electroporation or any other suitable transformation technology available in the art. Amyloid-reactive peptide-Fc fusion protein producing cell lines can then be selected and expanded and antibodies purified. The purified amyloid-reactive peptide-Fc fusion protein can then be analyzed by standard techniques such as SDS-PAGE or SEC.

Also provided is a host cell comprising a nucleic acid encoding any of the amyloid-reactive peptide-Fc fusion proteins described herein. Suitable host cells for cloning or expression of the amyloid-reactive peptide-Fc fusion protein-encoding vectors include prokaryotic or eukaryotic cells described herein. For example, the amyloid-reactive peptide-Fc fusion protein may be produced in bacteria, in particular when glycosylation and Fc effector function are not needed. For expression of polypeptides in bacteria, sec, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254) After expression, the amyloid-reactive peptide-Fc fusion protein may be isolated from the bacterial cell paste in a soluble fraction and can be further purified.

In some embodiments, the host cell comprises a vector comprising a nucleic acid(s) encoding a amyloid-reactive peptide-Fc fusion protein of the present disclosure.

Suitable host cells for the expression of glycosylated amyloid-reactive peptide-Fc fusion protein are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. Sec, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, sec, e.g., Yazaki and Wu, Methods in Molecular Biology, Vol. 248 (B.K.C. Lo, ed., Humana Press, Totowa, NJ), pp. 255-268 (2003).

Also provided herein are methods of making an amyloid-reactive peptide-Fc fusion protein of the present disclosure. In some embodiments, the method comprises culturing a host cell of the present disclosure under conditions suitable for expression of the vector encoding the amyloid-reactive peptide-Fc fusion protein. In some embodiments, the method further comprises recovering the amyloid-reactive peptide-Fc fusion protein. In some embodiments, protein recovery involves disrupting the host cell, for example by osmotic shock, sonication, or lysis. Once the cells are disrupted, cell debris is removed by centrifugation or filtration. The amyloid-reactive peptide-Fc fusion proteins can then be further purified. In some embodiments, an amyloid-reactive peptide-Fc fusion protein of the disclosure is purified by various methods of protein purification, for example, by chromatography (e.g., ion exchange chromatography, affinity chromatography, and size-exclusion column chromatography), centrifugation, differential solubility, or by any other standard technique for the purification of proteins. For example, in some embodiments, the amyloid-reactive peptide-Fc fusion protein is isolated and purified by appropriately selecting and combining affinity columns such as Protein A column (e.g., POROS Protein A chromatography) with chromatography columns (e.g., POROS HS-50 cation exchange chromatography), filtration, ultra-filtration, de-salting and dialysis procedures. In some embodiments, a amyloid-reactive peptide-Fc fusion protein is conjugated to marker sequences, such as a peptide to facilitate purification. An example of a marker amino acid sequence is a hexa-histidine peptide, which can bind to a nickel-functionalized agarose affinity column with micromolar affinity. As an alternative, a hemagglutinin “HA” tag, which corresponds to an epitope derived from the influenza hemagglutinin protein can be used.

The amyloid-reactive peptide-Fc fusion protein may be made by any technique known to those of skill in the art, including chemical synthesis or recombinant means using standard molecular biological techniques.

### IV. Methods of Treatment

Also provided herein are methods of treating an amyloid related disorder comprising administering an amyloid-reactive peptide-Fc fusion protein disclosed herein to an individual.

In some embodiments, provided a method of treating an amyloid disease comprising administering a therapeutically effective amount of any one of the amyloid-reactive peptide-Fc fusion protein described herein to an individual in need thereof.

In some embodiments, the amyloid deposits may contribute to the pathology of a disease. In other embodiments, the amyloid deposits may be indicative of amyloidosis or an amyloid-related disease in an individual. In some embodiments, the amyloid-reactive peptide-Fc fusion protein binds to amyloids in an individual with an amyloidosis. In some embodiments, the amyloidosis is localized to a specific tissue or organ system, such as the liver, the heart, or the central nervous system.

In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis, AL amyloidosis, AH amyloidosis, Aβ amyloidosis, ATTR amyloidosis, hATTR amyloidosis, ALect2 amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer. In some embodiments, the amyloid related disease is selected from the group consisting of AL, AH, Aβ2M, ATTR, transthyretin, AA, AApoAI, AApoAII, AApoAIV, AApoCII, AApoCII, AGel, ALys, ALEct2, AFib, ACys, ACal, AMed, AIAPP, APro, AIns, APrP, ASPC, AGal7, ACor, Aker, ALac, AOAPP, ASem1, AEnf, or Aβ amyloidosis. In some embodiments, treatment with the amyloid-reactive peptide-Fc fusion protein results in the clearance of amyloid. In some embodiments, the amyloid-reactive peptide-Fc fusion protein binds to amyloids associated with normal aging. In other embodiments, the amyloid-reactive peptide-Fc fusion protein is used in the diagnosis, treatment, or prognosis of an amyloidosis or amyloid-related disease in an individual.

In some embodiments, provided herein is a method of treating an amyloid related disorder in an individual comprising administering a fusion protein provided herein, wherein the individual has amyloid in the kidney, liver, and/or heart. In some embodiments, the individual has ALA deposits in the kidney. In some embodiments, the individual has ALκ deposits in the kidney. In some embodiments, the individual has ALA deposits in the liver. In some embodiments, the individual has ALκ deposits in the liver. In some embodiments, the individual has ATTR deposits in the heart. In some embodiments, the individual has ALκ deposits in the heart. In some embodiments, the individual has Alzheimer's disease. In some embodiments, the individual has tau fibrils or alpha synuclein aggregates. In some embodiments, the individual has Parkinson's disease. In some embodiments, the individual has fibrils in the spleen. In some embodiments, the fusion protein comprises a first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer. In some embodiments, the amyloid reactive peptide comprises an amino acid sequence having at least 85% sequence identity to any one of the amino acid sequences set forth as SEQ ID NOs: 1-13. In some embodiments, the first/and or second human Fc domain comprises a peptide spacer. In some embodiments, the spacer comprises an amino acid sequence set forth in any one of SEQ IDNO: 14-17.

In some embodiments, provided herein is a method of treating an amyloid related disorder in an individual comprising administering a fusion protein provided herein, wherein the amyloid related disorder is selected from the group consisting of AA amyloidosis, AL amyloidosis, and ATTR amyloidosis. In some embodiments, the fusion protein comprises a first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer. In some embodiments, the amyloid reactive peptide comprises an amino acid sequence having at least 85% sequence identity to any one of the amino acid sequences set forth as SEQ ID NOs: 1-13. In some embodiments, the first/and or second human Fc domain comprises a peptide spacer. In some embodiments, the spacer comprises an amino acid sequence set forth in any one of SEQ IDNO: 14-17. In some embodiments, the individual has amyloid deposits in the spleen, kidney, liver, and/or heart. In some embodiments, the fusion protein promotes phagocytosis by fixing complement.

In some embodiments, the amyloid-reactive peptide-Fc fusion protein is administered via an intradermal, subcutaneous, intramuscular, intracardiac, intravascular, intravenous, intra-ocular, intra-arterial, epidural, intraspinal, extracorporeal, intrathecal, intraperitoneal, intrapleural, intraluminal, intravitreal, intracavernous, intraventricular, intra-bone, intra-articular, intracellular, or pulmonary route.

In some embodiments, the amyloid-reactive peptide-Fc fusion protein is administered in sufficient amounts to induce phagocytosis of the amyloid by cells of the immune system (e.g., macrophages).

In some embodiments, the individual is a mammal such as primate, bovine, rodent, or pig. In some embodiments, the individual is a human.

Also provided herein are methods of targeting an amyloid deposit for clearance, comprising contacting an amyloid deposit with any one of the amyloid-reactive peptide-Fc fusion proteins described herein. In some embodiments, targeting the amyloid deposit for clearance results in clearance of the amyloid deposit. In some embodiments, clearance results from opsonization of the amyloid deposit. In some embodiments, the method results in phagocytosis of the amyloid. In some embodiments, the individual is a human. In some embodiments, the fusion protein comprises a first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer. In some embodiments, the amyloid reactive peptide comprises an amino acid sequence having at least 85% sequence identity to any one of the amino acid sequences set forth as SEQ ID NOs: 1-13. In some embodiments, the first/and or second human Fc domain comprises a peptide spacer. In some embodiments, the spacer comprises an amino acid sequence set forth in any one of SEQ IDNO: 14-17.

Also provided herein are methods of treating an individual having or suspected of having, an amyloid-based disease, comprising: determining whether the individual has an amyloid deposit by: detectably labeling any one of the amyloid-reactive peptide-Fc fusion proteins described herein, administering the labeled fusion protein to the individual, determining whether a signal associated with the detectable label can be detected from the individual; and, if the signal is detected, administering to the individual an amyloidosis treatment. In some embodiments, if a signal is not detected, monitoring the individual for a later development of an amyloid deposit. In some embodiments, the method further comprises determining the intensity of the signal and comparing the signal to a threshold value, above which the individual is determined to possess an amyloid deposit. In some embodiments, the amyloidosis treatment comprises administering any one of the amyloid-reactive peptide-Fc fusion proteins described herein to the individual. In some embodiments, the fusion protein comprises a first polypeptide comprising a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer. In some embodiments, the amyloid reactive peptide comprises an amino acid sequence having at least 85% sequence identity to any one of the amino acid sequences set forth as SEQ ID NOs: 1-13. In some embodiments, the first/and or second human Fc domain comprises a peptide spacer. In some embodiments, the spacer comprises an amino acid sequence set forth in any one of SEQ IDNO: 14-17.

In some embodiments, provided herein are methods of identifying an amyloid deposit in an individual comprising administering a detectably labeled fusion protein to the individual and detecting a signal from the fusion protein.

### V. Methods of Detection

Also provided herein are methods of identifying an amyloid deposit in an individual.

The method comprises obtaining a tissue sample from a subject, applying the peptide or fusion peptide to the tissue sample and detecting binding of the amyloid-reactive peptide-Fc fusion to the amyloid. Detecting the presence of amyloids may involve visualizing the binding of the peptide or fusion peptide to the amyloid using fluorescence, or standard histochemical techniques. The method may further comprise obtaining tissue sections from the tissue samples and staining the tissue sections and detecting the presence of amyloids in the tissue samples by visualizing the binding of the peptide to the amyloid using fluorescence, or standard histochemical techniques.

In some embodiments, provided is a method of identifying an amyloid deposit in an individual comprising detectably labeling any one of the amyloid-reactive peptide-Fc fusion proteins described herein, administering the fusion protein to the individual, and detecting a signal from the fusion protein. Any one of the detectably-labeled amyloid-reactive peptide-Fc fusion proteins described herein may be used. In some embodiments, the peptide-Fc fusion protein is radiolabeled. In some embodiments, the peptide-Fc fusion protein is radiolabeled with I-¹²⁵. In some embodiments, the amyloid-reactive peptide-Fc fusion protein is detected by SPECT/CT imaging, PET/CT imaging, gamma scintigraphy, or optical imaging. In some embodiments, the peptide-Fc fusion protein is fluorescently labeled. In some embodiments, the method further comprises determining the signal intensity. In some embodiments, the signal intensity is determined by a SPECT/CT scan or a microradiograph. In some embodiments, the fusion protein comprises a first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer. In some embodiments, the amyloid reactive peptide comprises an amino acid sequence having at least 85% sequence identity to any one of the amino acid sequences set forth as SEQ ID NOs: 1-13. In some embodiments, the first/and or second human Fc domain comprises a peptide spacer. In some embodiments, the spacer comprises an amino acid sequence set forth in any one of SEQ IDNO: 14-17.

In certain embodiments, the amyloid-reactive peptide-Fc fusion proteins of the present invention may be attached to imaging agents useful for imaging of amyloids in organs and tissues. For example, amyloid-reactive peptide-Fc fusion proteins of the present invention may be attached to an imaging agent, provided to a subject and the precise location of the amyloid may be determined by standard imaging techniques. Peptides that are non-selective for amyloids may be used as control for comparison. Thus, the biodistribution of the amyloid-reactive peptide-Fc fusion proteins of the present invention may be compared to the biodistribution of one or more non-selective or control peptides to provide even greater discrimination for detection and/or localization of amyloids.

Methods for imaging amyloids include but are not limited to magnetic resonance imaging (MRI), computed axial tomography (CAT) scanning, positron emission tomography (PET), ultrasonic imaging, x-rays, radionuclide imaging, single photon emission computed tomography (SPECT), and multiphoton microscopy.

To increase the sensitivity of scans, various contrast media may be used. The contrast media for scans may include all molecules that attenuate x-rays. For positron emission tomography and radionuclide imaging, radioisotopes may be used. All positron emitting isotopes are useful for positron emission tomography radionuclide imaging, and all γ-photon emitting isotopes are useful for radionuclide imaging for single photon emission computed tomography or scintigraphy imaging.

Contrast agents for ultrasonic imaging include positive agents and negative agents. Positive agents reflect the ultrasonic energy and thus they produce a positive (light) image. Correspondingly, negative agents enhance transmissibility or sonolucency and thus produce a negative (dark) image. A variety of substances—gases, liquids, solids, and combinations of these—has been investigated as potential contrast-enhancing agents. Examples of solid particle contrast agents disclosed in U.S. Pat. No. 5,558,854 include but not limited to IDE particles and SHU454. European Patent Application 0231091 discloses emulsions of oil in water containing highly fluorinated organic compounds for providing enhanced contrast in an ultrasound image. Emulsions containing perfluorooctyl bromide (PFOB) have also been examined as ultrasound imaging agents. U.S. Pat. No. 4,900,540 describes the use of phospholipid-based liposomes containing a gas or gas precursor as a contrast-enhancing agent.

Imaging agents may be attached to amyloid-reactive peptide-Fc fusion proteins of the present invention using known methods. Certain attachment methods involve the use of a metal chelate complex employing, for example, an organic chelating agent such a DTPA. Acceptable chelates are known in the field. They include but are not limited to 1,4,7,10-tetraazacyclododecane-N,N′,N″,N′″-tetraacetic acid (DOTA); 1,4,7,10-tetraazacyclododecane-N,N′,N″-triacetic acid (DO3A); 1,4,7-tris(carboxymethyl)-10-(2-hydroxypropyl)-1,4,7,10-tetraazacyclododecane (HP-DO3A); diethylenetriaminepentaacetic acid (DPTA); and many others.

Several classes of compounds have potential as MRI contrast agents. These classes include supraparamagnetic iron oxide particles, nitroxides, and paramagnetic metal chelates (Mann et al., 1995). A strong paramagnetic metal is preferred. Normally, paramagnetic lanthanides and transition metal ions are toxic in vivo. Thus, it is necessary to incorporate these compounds into chelates with organic ligands. The amyloid-reactive peptide-Fc fusion proteins of the present invention may be used to enhance the targeting of such chelated metals to amyloids, which allows for the reduction in the total dose of imaging composition otherwise required.

Paramagnetic metals of a wide range are suitable for chelation. Suitable metals include those having atomic numbers of 22-29 (inclusive), 42, 44 and 58-70 (inclusive), and having oxidation states of 2 or 3. Examples of such metals include but are not limited to chromium (III), manganese (II), iron (II), cobalt (II), nickel (II), copper (II), praseodymium (III), neodymium (III), samarium (III), gadolinium (III), terbium (III), dysprosium (III), holmium (III), erbium (III), ytterbium (III), and vanadium (II). Ions useful in other contexts, such as X-ray imaging, include but are not limited to lanthanum (III), gold (III), lead (II), and especially bismuth (III).

Among the radioisotopes that can be used to label amyloid-reactive peptide-Fc fusion proteins of the present invention that are suitable for localization studies are gamma-emitters, positron-emitters, X-ray-emitters and fluorescence-emitters. Appropriate radioisotopes for labeling peptides and fusion proteins include astatine²¹¹, bromine⁷⁶, ¹⁴-carbon, ¹¹carbon, ⁵¹chromium, ³⁶chlorine, ⁵⁷cobalt, ⁵⁸cobalt, copper⁶⁷, copper⁶⁴, ¹⁵² europium, fluorine¹⁸, gallium⁶⁷, Gallium⁶⁸, ³hydrogen, iodine¹²³, iodine¹²⁴, iodine¹²⁵, iodine¹²⁶, iodine¹³¹, indium¹¹¹, indium^{113m}, ⁵⁹iron, ¹⁷⁷lutetium, mercury¹⁰⁷, mercury²⁰³, ³²phosphorus, rhenium¹⁸⁶, rhenium¹⁸⁸, ruthenium⁹⁵, ruthenium⁹⁷, ruthenium¹⁰³, ruthenium¹⁰⁵, rhenium^{99m}, rhenium¹⁰⁵, rhenium¹⁰¹, ⁷⁵selenium, ³⁵sulphur, technitium^{99m}, tellurium^{121m}tellurium^{122m}, tellurium^{125m}, thulium¹⁶⁵, thulium¹⁶⁷, thulium¹⁶⁸, and yttrium⁹⁰. The halogens may be used more or less interchangeably as labels. The gamma-emitters, iodine¹²³ and technetium^{99m}, may also be used because such radiometals are detectable with a gamma camera and have favorable half lives for imaging in vivo. The positron-emitters ¹⁸-fluorine or ¹²⁴iodine which are suitable for PET imaging and have suitable half lives for peptide imaging may also be used. Peptides and fusion peptides of the present invention may be labeled with indium¹¹¹ or technetium^{99m} via a conjugated metal chelator, such as DTPA (diethlenetriaminepentaacetic acid) or covalently and directly to the flanking peptide that contains a Cys residue.

Radioactively labeled amyloid-reactive peptide-Fc fusion proteins of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Peptides or fusion peptides according to the invention may be labeled with technetium^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent, such as SnCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA), as mentioned earlier.

Other useful labels include fluorescent labels, chromogenic labels, and biotin labels. Fluorescent labels, include but are not limited to rhodamine, fluorescein isothiocyanate, fluorescein sodium, renographin, and Texas Red sulfonyl chloride. In certain embodiments, the peptides and fusion peptides of the present invention may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Amyloid-reactive peptide-Fc fusion proteins of the present invention also may be reacted with an enzyme in the presence of a coupling agent such as glutaraldehyde or periodate. Conjugates with fluorescein markers are prepared in the presence of these coupling agents or by reaction with an isothiocyanate.

The amyloid-reactive peptide-Fc fusion proteins of the present invention serve as agents that target and bind amyloids to enable detection of amyloids. The peptides and fusion peptides of the present invention can be used to determine whether a subject has amyloid and whether a subject is suffering from amyloidosis or amyloid mediated condition.

In some embodiments, the present invention provides a method for detecting amyloids in a subject. The method comprises administering a pharmaceutical composition comprising an effective amount of one or more peptides or fusion peptides of the present invention to a subject and detecting the peptides or fusion peptides bound to the amyloids. The amyloid-reactive peptide-Fc fusion proteins may be labeled with an imaging agent, such as a radioisotope. The amyloid-reactive peptide-Fc fusion proteins has specific binding affinity for the deposits and the binding is detectable. The binding of the amyloid-reactive peptide-Fc fusion proteins peptides to the amyloids may be detected by MRI, CAT scan, PET imaging, ultrasound imaging, SPECT imaging, X-ray imaging, fluorescence imaging, or radionuclide imaging.

In some embodiments, the individual has one or more risk factors associated with an amyloid related disease. In some embodiments, the individual has one or more symptoms of an amyloid related disease.

With regard to amyloidosis, such labeling, for example, can be used to diagnose the presence of amyloid, to determine the amyloid protein load, to monitor the ability of the amyloid-reactive peptide-Fc fusion proteins to bind amyloid in a particular individual, to monitor the progression of amyloidosis, and/or to monitor an individual's response to an amyloid treatment (including treatments associated with the administration of the amyloid-reactive peptide-Fc fusion proteins to the individual). For example, amyloid-reactive peptide-Fc fusion proteins are labeled with a detectable label as described herein and thereafter administered to an individual that is suffering from, or suspected to be suffering from, an amyloid-based disease (e.g., amyloidosis, monoclonal gammopathy of unknown significance (MGUS), multiple myeloma (MM), or related plasma cell diseases). Thereafter, the individual can be imaged, for example, to detect the presence of the detectably-labeled amyloid-reactive peptide-Fc fusion proteins.

In certain example embodiments, the signals from the detectably-labeled amyloid-reactive peptide-Fc fusion proteins can be quantified, thereby providing an indication of the level of amyloid deposit in the individual. For example, the signal intensity may be compared to a standard signal threshold, above which amyloidosis is present but below which amyloidosis is absent or at a low level. The individual can be diagnosed as having amyloid, in which case a treatment can be administered, such as such as chemotherapy, corticosteroid medicines (lenalidomide or thalidomide) and/or bortezomib (Velcade). Additionally or alternatively, the amyloid-reactive peptide-Fc fusion proteins described herein can be administered to the individual in an effort to treat the individual as described herein. In certain example embodiments, the individual may be stratified into one or more groups, such as a low amyloid load, medium amyloid load, or high amyloid load, and then treated accordingly. To monitor treatment progress, the individual may be re-administered the detectably-labeled amyloid-reactive peptide-Fc fusion proteins, and hence reassessed for their amyloid load.

### EMBODIMENTS

1. An amyloid-reactive peptide-Fc fusion protein comprising a first polypeptide and a second polypeptide, wherein the first polypeptide comprises a first amyloid-reactive peptide linked to the N-terminus or C-terminus of a first human Fc domain, wherein the second polypeptide comprises a second amyloid-reactive peptide linked to the N-terminus or C-terminus of a second human Fc domain, and wherein the first and the second human Fc domains form a dimer.
2. The amyloid-reactive peptide-Fc fusion protein of embodiment 1, wherein the first and second-amyloid reactive peptides are linked to the C-terminus of the first and second human Fc domains.
3. The amyloid-reactive peptide-Fc fusion protein of embodiment 1 or 2, wherein the first and/or the second amyloid-reactive peptide comprises an amino acid sequence having at least 85% sequence identity to any one of the amino acid sequences set forth as SEQ ID NOs: 1-13.
4. The amyloid-reactive peptide-Fc fusion protein of any one of embodiments 1-3, wherein the first and/or second human Fc domain is a human IgG1, IgG2, or IgG4 Fc.
5. The amyloid-reactive peptide-Fc fusion protein of any one of embodiments 1-4, wherein the first and/or second human Fc domain is a human IgG1 Fc.
6. The amyloid-reactive peptide-Fc fusion protein of any one of embodiments 1-5, wherein the first and/or second human Fc domain comprises an amino acid sequence set forth in SEQ ID NO: 18.
7. The amyloid-reactive peptide-Fc fusion protein of any one of embodiments 1-6, wherein the first and/or second amyloid-reactive peptide is linked to the first and/or second human Fc domain via a spacer.
8. The amyloid-reactive peptide-Fc fusion protein of embodiment 7, wherein the spacer is a peptide spacer.
9. The amyloid-reactive peptide-Fc fusion protein of embodiment 8, wherein the spacer comprises an amino acid sequence set forth in any one of SEQ ID NOs: 14-17.
10. The amyloid-reactive peptide-Fc fusion protein of any one of embodiments 1-9, wherein the first polypeptide comprises, from N- to C-terminus, a first human Fc domain, a first spacer, and a first amyloid-reactive peptide, and the second polypeptide comprises, from N- to C-terminus a second human Fc domain, a second spacer, and a second amyloid-reactive peptide.
11. The amyloid-reactive peptide-Fc fusion protein of embodiment 10, wherein the amyloid-reactive peptide comprises the amino acid sequence set forth in SEQ ID NO:2 or SEQ ID NO: 13.
12. The amyloid-reactive peptide-Fc fusion protein of embodiment 10, wherein the amyloid-reactive peptide comprises the amino acid sequence set forth in SEQ ID NO:2 and the spacer comprises the amino acid sequence set forth in SEQ ID NO:14.
13. The amyloid-reactive peptide-Fc fusion protein of embodiment 10, wherein the amyloid reactive peptide comprises the amino acid sequence set forth in SEQ ID NO:13 and the spacer comprises the amino acid sequence set forth in SEQ ID NO: 14.
14. The fusion protein of embodiment 10, wherein the amyloid-reactive peptide comprises the amino acid sequence set forth in SEQ ID NO: 2 and the spacer comprises the amino acid sequence set forth in SEQ ID NO:17.
15. The amyloid-reactive peptide-Fc fusion protein of any one of embodiments 1-10, wherein
   i) the first polypeptide and/or second polypeptide comprises an amino acid sequence set forth in SEQ ID NO: 20;
   ii) the first polypeptide and/or second polypeptide comprises an amino acid sequence set forth in SEQ ID NO: 21;
   iii) the first polypeptide and/or second polypeptide comprises an amino acid sequence set forth in SEQ ID NO: 22.
16. The amyloid-reactive peptide-Fc fusion protein of embodiment 15, wherein the first polypeptide comprises an amino acid sequence set forth in SEQ ID NO: 20, and the second polypeptide comprises an amino acid sequence set forth in SEQ ID NO: 20.
17. The amyloid-reactive peptide-Fc fusion protein of any one of embodiments 1-9, wherein the first polypeptide comprises, from N- to C-terminus, a first amyloid-reactive peptide, a first spacer, and a first human Fc domain and the second polypeptide comprises, from N- to C-terminus a second amyloid reactive peptide, a second spacer, and a second human Fc domain.
18. The amyloid-reactive peptide-Fc fusion protein of embodiments 16, wherein the amyloid-reactive peptide comprises the amino acid sequence set forth in SEQ ID NO:2 or SEQ ID NO: 13.
19. The amyloid-reactive peptide-Fc fusion protein of embodiment 17 or 18, wherein the amyloid reactive peptide comprises the amino acid sequence set forth in SEQ ID NO:2 and the spacer comprises the amino acid sequences set forth in SEQ ID NO:14.
20. The amyloid-reactive peptide-Fc fusion protein of embodiments 17, wherein the first and/or second polypeptide comprises the amino acid sequence set forth in SEQ ID NO:19.
21. The amyloid-reactive peptide-Fc fusion protein of any one of embodiments 1-20, wherein the first and second polypeptides comprise the same amino acid sequence.
22. The amyloid-reactive peptide-Fc fusion protein of any one of embodiments 1-15 and 17-21 wherein the first and second polypeptide comprise different amino acid sequences.
23. The amyloid-reactive peptide-Fc fusion protein of any one of embodiments 1-22, wherein the amyloid-reactive peptide-Fc fusion protein binds to rVλ6Wil, Aβ, Aβ(1-40), IAAP, ALκ, ALλ, or ATTR amyloid.
24. The amyloid-reactive peptide-Fc fusion protein of any one of embodiments 1-23, wherein the fusion protein is conjugated to a detectable label.
25. The amyloid-reactive peptide-Fc fusion protein of embodiment 23, wherein the detectable label is selected from the group consisting of a fluorescent label and a radioactive label.
26. A pharmaceutical composition comprising the amyloid-reactive peptide-Fc fusion protein of any one of embodiments 1-24.
27. Nucleic acid(s) encoding the amyloid-reactive peptide-Fc fusion protein of any one of embodiments 1-25.
28. A vector comprising the nucleic acid(s) of embodiment 26.
29. A host cell comprising the vector of embodiment 28.
30. The host cell of embodiment 29, wherein the host cell is a mammalian cell, optionally a Chinese hamster ovary (CHO) cell.
31. A method of making an amyloid-reactive peptide-Fc fusion protein comprising culturing the host cell of embodiment 29 or 30 under conditions suitable for expression of the vector encoding the amyloid-reactive peptide-Fc fusion protein.
32. The method of embodiment 31, wherein the method further comprises recovering the amyloid-reactive peptide-Fc fusion protein.
33. A method of treating an amyloid disease comprising administering a therapeutically effective amount of the amyloid-reactive peptide-Fc fusion protein of any one of embodiments 1-24 to an individual in need thereof.
34 The method of embodiment 33, wherein the amyloid related disease is systemic or localized amyloidosis.
35. The method of embodiment 33, wherein the amyloid related disease is selected from the group consisting of AL, AH, Aβ2M, ATTR, transthyretin, AA, AApoAI, AApoAII, AGel, ALys, ALEct2, AFib, ACys, ACal, AMed, AIAPP, APro, AIns, APrP, Parkinson's disease Alzheimer's disease or Aβ amyloidosis.
36. The method of any one of embodiments 33-35, wherein the treatment with the amyloid-reactive peptide-Fc fusion protein results in the clearance of amyloid.
37. A method of targeting an amyloid deposit for clearance, comprising contacting an amyloid deposit with the amyloid-reactive peptide-Fc fusion protein of any one of embodiments 1-25.
38. The method of embodiment 37, wherein targeting the amyloid deposit for clearance results in clearance of the amyloid deposit.
39. The method of embodiment 37 or 38, wherein clearance results from opsonization of the amyloid deposit.
40. A method of treating an individual having an amyloid-based disease or suspected of having an amyloid-based disease, comprising:
   determining whether the individual has an amyloid deposit by:
      detectably labeling the amyloid-reactive peptide-Fc fusion protein of any one of embodiments 1-25,
      administering the labeled amyloid-reactive peptide-Fc fusion protein to the individual,
      determining whether a signal associated with the detectable label can be detected from the individual; and,
      if the signal is detected, administering to the individual an amyloidosis treatment.
41. The method of embodiment 40, wherein, if a signal is not detected, monitoring the individual for a later development of an amyloid deposit.
42. The method of embodiment 40 or 41, further comprising determining the intensity of the signal and comparing the signal to a threshold value, above which the individual is determined to possess an amyloid deposit.
43. The method of any of embodiments 40-42, wherein the amyloidosis treatment comprises administering the amyloid-reactive peptide-Fc fusion protein of any one of embodiments 1-25 to the individual.
44. A method of identifying an amyloid deposit in an individual, comprising detectably labeling the amyloid-reactive peptide-Fc fusion protein of any one of embodiments 1-25, administering the fusion protein to the individual, and detecting a signal from the fusion protein.
45. The method of any of embodiments 40-44, wherein the individual is determined to be amyloid free or suffering from monoclonal gammopathy of unknown significance (MGUS), multiple myeloma (MM), or one or more related plasma cell diseases.
46. The method of embodiment 44 or 45, wherein the amyloid-reactive peptide-Fc fusion protein is detectably labeled.
47. The method of any one of embodiments 40-43 and 46, wherein the amyloid-reactive peptide-Fc fusion protein is detectably labeled with a radionuclide or a fluorophore.
48. The method of embodiment 47, wherein the radionuclide is I-123, I-124, F-18, ZR-89, or Tc-99m.
49. The method of any one of embodiments 40-43 and 46-48, wherein the amyloid-reactive peptide-Fc fusion protein is detected by SPECT/CT imaging, PET/CT imagining, gamma scintigraphy, or optical imaging.
50. The method of any one of embodiments 33-49, wherein the individual is a human.
51. The amyloid-reactive peptide-Fc fusion protein of any one of embodiments 1-25, wherein the amyloid-reactive peptide-Fc first polypeptide and the second polypeptide are covalently linked by a disulfide bond in the Fc domain.
1A. A fusion protein comprising a first polypeptide and a second polypeptide, wherein the first polypeptide comprises a first amyloid-reactive peptide linked to the C-terminus of a first human Fc domain, wherein the second polypeptide comprises a second amyloid-reactive peptide linked to the C-terminus of a second human Fc domain, and wherein the first and the second human Fc domains form a dimer.
2A. The fusion protein of embodiment 1A, wherein the first and/or the second amyloid-reactive peptide comprises an amino acid sequence having at least 85% sequence identity to any one of the amino acid sequences set forth as SEQ ID NOs: 1-13.
3A. The fusion protein of embodiment 1A or embodiment 2A, wherein the first and/or second human Fc domain is a human IgG1, IgG2, or IgG4 Fc.
4A. The fusion protein of any one of embodiments 1A-3A, wherein the first and/or second human Fc domain is a human IgG1 Fc.
5A. The fusion protein of any one of embodiments 1A-4A, wherein the first and/or second human Fc domain comprises an amino acid sequence set forth in SEQ ID NO: 18.
6A. The fusion protein of any one of embodiments 1A-5A, wherein the first and/or second amyloid-reactive peptide is linked to the first and/or second human Fc domain via a spacer.
7A. The fusion protein of embodiment 6A, wherein the spacer is a peptide spacer.
8A. The fusion protein of embodiment 7A, wherein the spacer comprises an amino acid sequence set forth in any one of SEQ ID NOs: 14-17.
9A. The fusion protein of any one of embodiments 6A-8A, wherein the first polypeptide comprises, from N- to C-terminus, a first human Fc domain, a first spacer, and a first amyloid-reactive peptide, and the second polypeptide comprises, from N- to C-terminus a second human Fc domain, a second spacer, and a second amyloid-reactive peptide.
10A. The fusion protein of any one of embodiments 1A-9A, wherein the first polypeptide comprises an amino acid sequence set forth in SEQ ID NO: 20, and the second polypeptide comprises an amino acid sequence set forth in SEQ ID NO: 20.
11A. The fusion protein of any one of embodiments 1A-10A, wherein the fusion protein binds to rVλ6Wil, Aβ, Aβ(1-40), IAAP, ALκ4, Alλ1, or ATTR amyloid.
12A. The fusion protein of any one of embodiments 1A-11A, wherein the fusion protein is conjugated to a detectable label.
13A. A pharmaceutical composition comprising the fusion protein of any one of embodiments 1A-12A.
14A. Nucleic acid(s) encoding the fusion protein of any one of embodiments 1A-12A.
15A. A vector comprising the nucleic acid(s) of embodiment 14A.
16A. A host cell comprising the vector of embodiments 15A.
17A. The host cell of embodiments 16A, wherein the host cell is a mammalian cell, optionally a Chinese hamster ovary (CHO) cell.
18A. A method of making a fusion protein comprising culturing the host cell of embodiment 16A or 17A under conditions suitable for expression of the vector encoding the fusion protein.
19A. The method of embodiment 18A, wherein the method further comprises recovering the fusion protein.
20A. A method of treating an amyloid disease comprising administering a therapeutically effective amount of the fusion protein of any one of embodiments 1A-12A to an individual in need thereof.
21A. The method of embodiment 20A, wherein the amyloid related disease is systemic or localized amyloidosis.
22A. The method of embodiment 20A, wherein the amyloid related disease is selected from the group consisting of AL, AH, Aβ2M, ATTR, transthyretin, AA, AApoAI, AApoAII, AGel, ALys, ALEct2, AFib, ACys, ACal, AMed, AIAPP, APro, AIns, APrP, or Aβ amyloidosis.
23A. The method of any one of embodiments 20A-22A, wherein the treatment with the fusion protein results in the clearance of amyloid.
24A. A method of targeting an amyloid deposit for clearance, comprising contacting an amyloid deposit with the fusion protein of any one of embodiments 1A-12A.
25A. The method of embodiment 24A, wherein targeting the amyloid deposit for clearance results in clearance of the amyloid deposit.
26A. The method of embodiment 24A or 25A, wherein clearance results from opsonization of the amyloid deposit.
27A. The method of any one of embodiments 20A-26A, wherein the individual is a human.
28A. A method of treating an individual suffering from, or suspected to be suffering from, an amyloid-based disease, comprising:
   determining whether the individual has an amyloid deposit by:
      detectably labeling the fusion protein of any one of embodiments 1A-12A,
      administering the labeled fusion protein to the individual,
      determining whether a signal associated with the detectable label can be detected from the individual; and,
      if the signal is detected, administering to the individual an amyloidosis treatment.
29A. The method of embodiment 28A, wherein, if a signal is not detected, monitoring the individual for a later development of an amyloid deposit.
30A. The method of embodiment 29A, further comprising determining the intensity of the signal and comparing the signal to a threshold value, above which the individual is determined to possess an amyloid deposit.
31A. The method of any of embodiments 28A-30A, wherein the amyloidosis treatment comprises administering the fusion protein of any one of embodiments 1A-12A to the individual.
32A. A method of identifying an amyloid deposit in an individual, comprising detectably labeling the fusion protein of any one of embodiments 1A-12A, administering the fusion protein to the individual, and detecting a signal from the fusion protein.
33A. The method of any of embodiments 28A-32A, wherein the individual is determined to be amyloid free or suffering from monoclonal gammopathy of unknown significance (MGUS), multiple myeloma (MM), or one or more related plasma cell diseases.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter. The attached figures are meant to be considered as integral parts of the specification and description of the disclosure.

### Example 1. Production of Peptide-Fc Constructs

The following example describes the production of amyloid-reactive peptide-Fc fusion protein constructs. The structures of exemplary constructs are provided in FIG. 1. In one construct, termed Fcp5R NV1, the p5R peptide was fused to the N-terminus of a first and second Fc domain via a short, rigid spacer (VSPSV, SEQ ID NO: 15), as shown in the top row of FIG. 1. In a second construct, termed Fcp5R CV1, the p5R peptide was fused to the C-terminus of a first and second Fc domain via a short, rigid spacer (VSPSV, SEQ ID NO: 15), as shown in the second row of FIG. 1. The amino acid sequences of Fcp5R NV1 and Fcp5R CV1 are provided in Table 3, below.

TABLE 3
Amino acid sequences of
peptide-Fc constructs

| | | SEQ |
|---|---|---|
| | | ID |
| Construct | Amino acid sequence | NO |
| Fcp5R | APGGGRAQRAQARQARQAQR | SEQ |
| NV1 or | AQRAQARQARQVSPSVDKTH | ID |
| hFc1NV1 | TCPPCPAPELLGGPSVFLFP | NO: |
| | PKPKDTLMISRTPEVTCVVV | 19 |
| | DVSHEDPEVKENWYVDGVEV | |
| | HNAKTKPREEQYNSTYRVVS | |
| | VLTVLHQDWLNGKEYKCKVS | |
| | NKALPAPIEKTISKAKGQPR | |
| | EPQVYTLPPSRDELTKNQVS | |
| | LTCLVKGFYPSDIAVEWESN | |
| | GQPENNYKTTPPVLDSDGSF | |
| | FLYSKLTVDKSRWQQGNVFS | |
| | CSVMHEALHNHYTQKSLSLS | |
| | PGK | |
| Fcp5R | APGGGSVSDKTHTCPPCPAP | SEQ |
| CV1 or | ELLGGPSVFLFPPKPKDTLM | ID |
| hFc1CV1 | ISRTPEVTCVVVDVSHEDPE | NO: |
| | VKFNWYVDGVEVHNAKTKPR | 20 |
| | EEQYNSTYRVVSVLTVLHQD | |
| | WLNGKEYKCKVSNKALPAPI | |
| | EKTISKAKGQPREPQVYTLP | |
| | PSRDELTKNQVSLTCLVKGF | |
| | YPSDIAVEWESNGQPENNYK | |
| | TTPPVLDSDGSFFLYSKLTV | |
| | DKSRWQQGNVFSCSVMHEAL | |
| | HNHYTQKSLSLSPGKVSPSV | |
| | RAQRAQARQARQAQRAQRAQ | |
| | ARQARQ | |

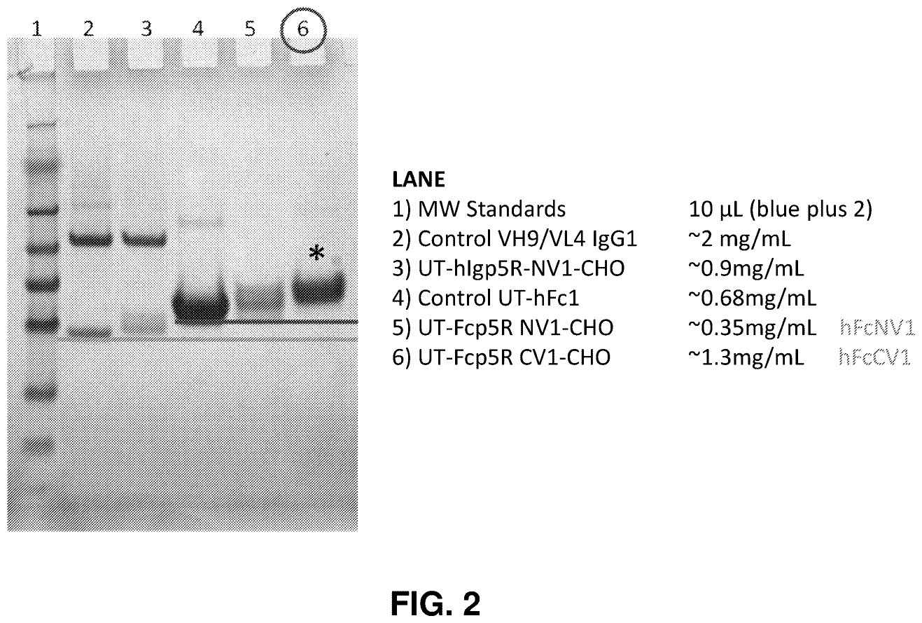

A sodium dodecyl sulfate polyacrylamide gel electrophoresis (SDS-PAGE) analysis was performed to compare preparations of the peptide-Fc fusion constructs to peptide-antibody fusion constructs. The fusion constructs were produced in Chinese hamster ovary (CHO) cells with 2% fetal bovine serum (FBS). Samples were reduced and boiled. 4-12% bis-tris gels were used with a 2-(N-morpholino)ethanesulfonic acid (MES) buffer, and Coomassie blue stain was used to detect the proteins. As shown in FIG. 2, the Fcp5R CV1 construct (lane 6) was larger in size than the Fcp5R NV1 construct (lane 5). Without wishing to be bound by theory, it is believed that this method of production resulted in constructs that were susceptible to cleavage of the amyloid binding peptide (in the case of Fcp5R NV1, lane 5) or resistant to peptide cleavage (in the case of Fcp5R CV1, lane 6). The data indicated that the position of the peptide on the Fc domain can protect it from proteolysis during production.

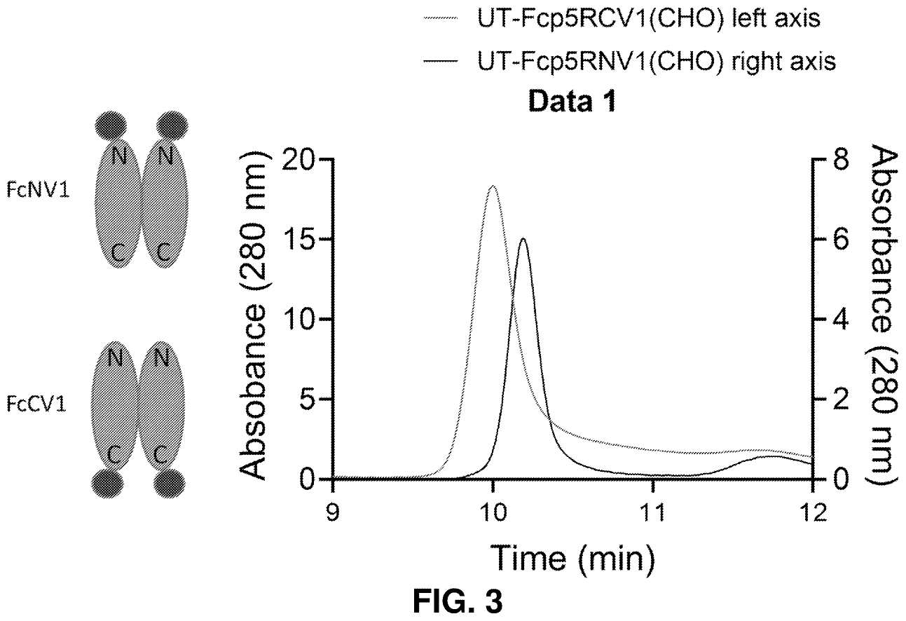

Size exclusion chromatography (SEC) was performed to further analyze Fcp5R NV1 and Fcp5R CV1. As shown in FIG. 3, Fcp5R NV1 and Fcp5R CV1 eluted from the column at different times, indicating that the two constructs were different size. The Fcp5R CV1 eluted before NV1, indicating that it had a higher molecular weight consistent with the observations from the SDS-PAGE analysis (FIG. 2) that the peptide was resistant to proteolytic degradation during production of the reagent. In contrast, the Fcp5R NV1 was susceptible to proteolytic cleavage.

### Example 2. Biodistribution of Peptide-Fc in Mice

The following example describes the radioiodination of Fcp5R CV1, and the administration of radiolabeled Fcp5R CV1 to AA amyloidosis mice.

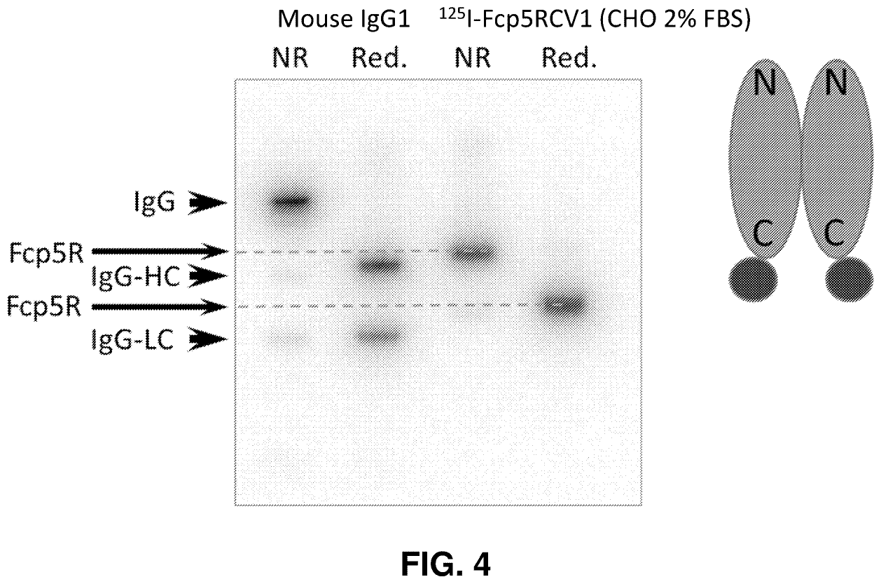

Fcp5R CV1 was radiolabeled with I-¹²⁵. The antibody 11-1F4 served as a control for the radiolabeling reaction. As shown in FIG. 4, Fcp5R CVI produced by HD CHO cells grown in 2% FBS was readily radiolabeled with I-¹²⁵ and eluted with the blue dextran. Recovery from the column and tubes was excellent. The SDS-PAGE gel indicated that no aggregates were formed during the radiolabeling procedure and that the preparation was of high purity and high radiopurity (i.e., there was no evidence of free radioiodide in the SDS-PAGE gel).

¹²⁵I-Fcp5R CV1 was administered to mice with systemic AA amyloidosis. The AA amyloidosis mice model was generated by IV administration of 0.1 mg of isolated amyloid enhancing factor (AEF, Axelrad et al., Lab Invest (1982)47: 139-146) in 100 μL of sterile phosphate-buffered saline (PBS) in H2-Ld-huIL-6 Tg Balb/c transgenic mice that constitutively express the human interleukin-6 transgene. Mice used in these studies were 4-6 weeks post induction of amyloidosis. The AA mice model is characterized by extensive sinusoidal amyloid deposits in liver, initial and massive perifollicular amyloid deposits in spleen, and later amyloid deposits in pancreas, kidney, adrenal glands, intestine, and scant interstitial cardiac amyloid deposition.

¹²⁵I-Fcp5R CV1 (˜10 μg, ˜100 μCi) was administered by intravenous tail vein injection to mice with AA amyloidosis, and the biodistribution of ¹²⁵I-Fcp5R CV1 was detected at time points post injection. Specifically, groups of AA mice (n=4 per group) were injected with ¹²⁵I labeled Fcp5R CV1, and then euthanized at 1, 4, or 24 hours post injection. Samples of spleen, pancreas, left and right kidney, liver, heart, muscle, stomach, upper and lower intestine, and lung tissue were harvested from AA mice after euthanasia. Each sample was placed into a tared plastic vial and weighed, and the ¹²⁵I radioactivity was measured by using an automated Wizard 3 gamma counter (1480 Wallac Gamma Counter, PERKIN ELMER®). The biodistribution data were expressed as percentage of injected dose per gram of tissue (% ID/g). In addition, samples of each tissue were fixed in 10% buffered formalin for 24 hours and embedded in paraffin for histology and autoradiography. For autoradiography, 4- to 6-μm thick sections were cut from formalin-fixed, paraffin-embedded blocks onto Plus microscope slides (FISHER SCIENTIFIC®), dipped in NTB2 emulsion (EASTMAN KODAK®), stored in the dark, and developed after a 96 hour exposure. Each section was counterstained with hematoxylin.

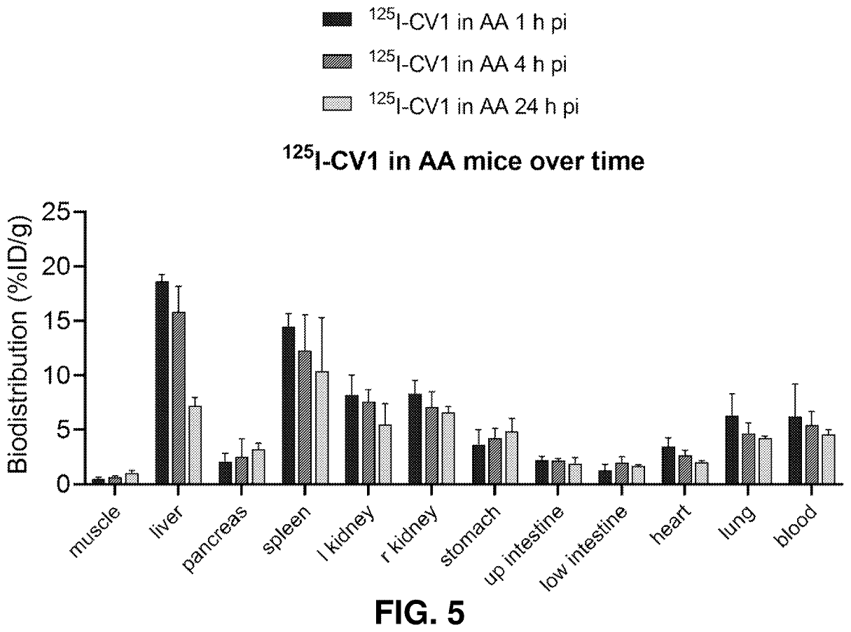

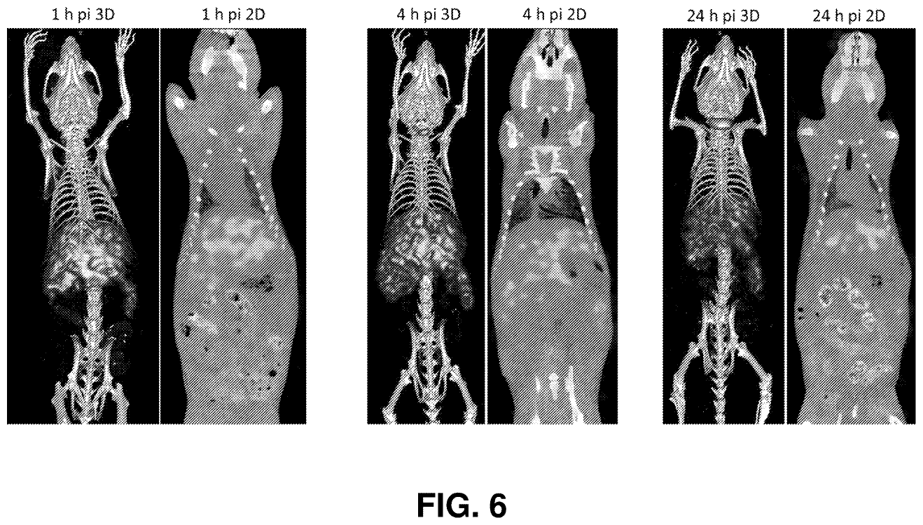

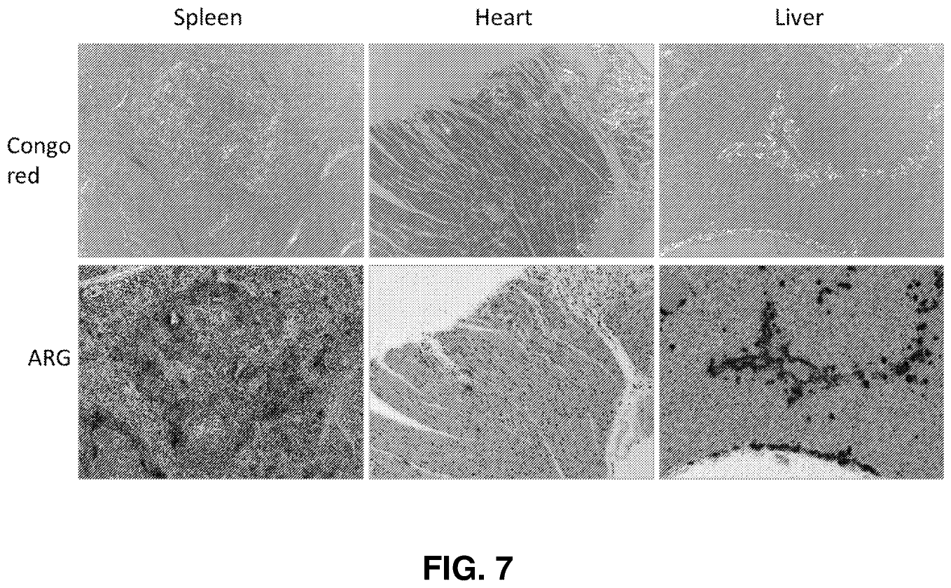

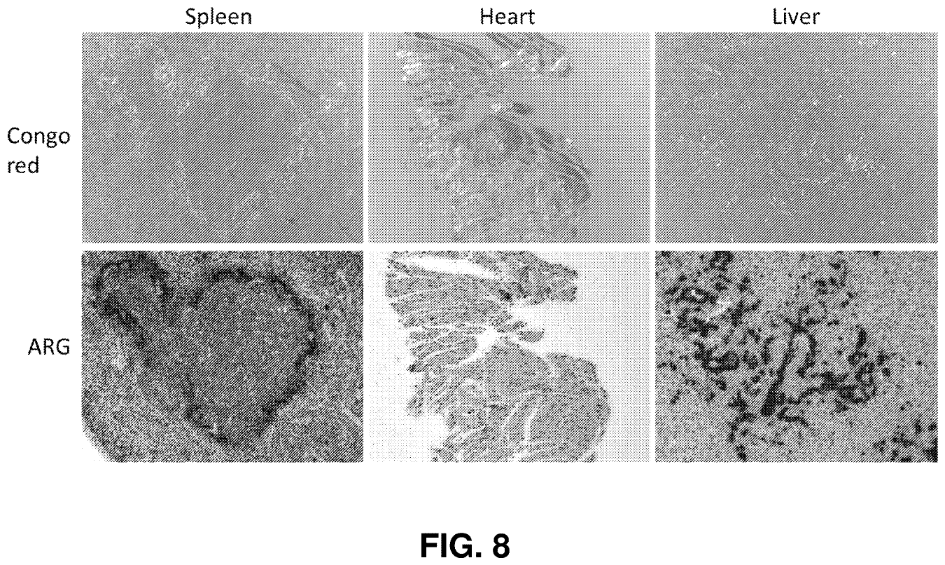

As shown in FIGS. 5-8, ¹²⁵I-Fcp5R CV1 was detected in the mice, in particular in the liver and the spleen, the major sites of amyloid deposition in this mouse model. Additional uptake of the tracer was observed in the kidneys (FIG. 5). The binding to amyloid deposits in the liver and spleen was visualized using small animal SPECT/CT imaging which demonstrated heatosplenic uptake of the radioiodinated Fcp5R CV1 for more than 24 hours post injection (FIG. 6). Specific binding to amyloid deposits in the heart, liver and spleen was demonstrated using microautoradiography (FIGS. 7-8). In the microautoradiographs (ARG), the presence of radiolabeled Fcp5R CV1 was indicated by the deposition of black silver grains. The distribution of the silver grains, and therefore ¹²⁵I-Fcp5R CVI correlated with the distribution of amyloid shown in Congo red-stained consecutive tissue sections (Congo red). The specific reactivity of ¹²⁵I-Fcp5R CV1 with amyloid in these tissues was observed also at later time points (24 hours) post injection (FIG. 8).

### Example 3. Binding and Phagocytosis of rVλ6Wil Fibrils

The ability of the peptide-Fc fusion proteins to promote amyloid fibril phagocytosis was studied using the pHrodo red-labeled rVλ6Wil fibrils system.

Human THP1 cells (10⁶ cells/well) were coated onto the wells of a 24-well tissue culture-treated plate. An aliquot of 50 ng/ml phorbol myristate acetate (PMA) was added and the cells incubated for 24 hours at 37° C. in a 5% CO₂ incubator. After 24 hours, the culture medium containing PMA is carefully removed and replaced with complete DMEM-F12 medium and the cells allowed to rest for a minimum of 48 hours. To perform the phagocytosis assay, media is removed from the wells and following a rinse with Dulbecco's PBS and an aliquot of 500 μL of RPMI added to each well. The Fcp5R variants, or control hFc1 are mixed with pHrodo red-labeled fibrils at the appropriate concentration before begin added to the cells in the 24-well plate. After gentle mixing, the plate is incubated for 1 hour at 37° C. in a 5% CO₂ incubator to facilitate phagocytosis. At the end of 1 hour incubation the fluorescence emission from the pHrodo red fluorophore was imaged using an epifluorecent microscope using the 4× objective and red fluorescence filter. Four images were captured for each well to ensure all areas of the well are covered and represented without any bias. The amount of fluorescence in each image was quantified using image segmentation and quantitation (Image ProPlus). The fluorescence units were measured as digital spectral counts.

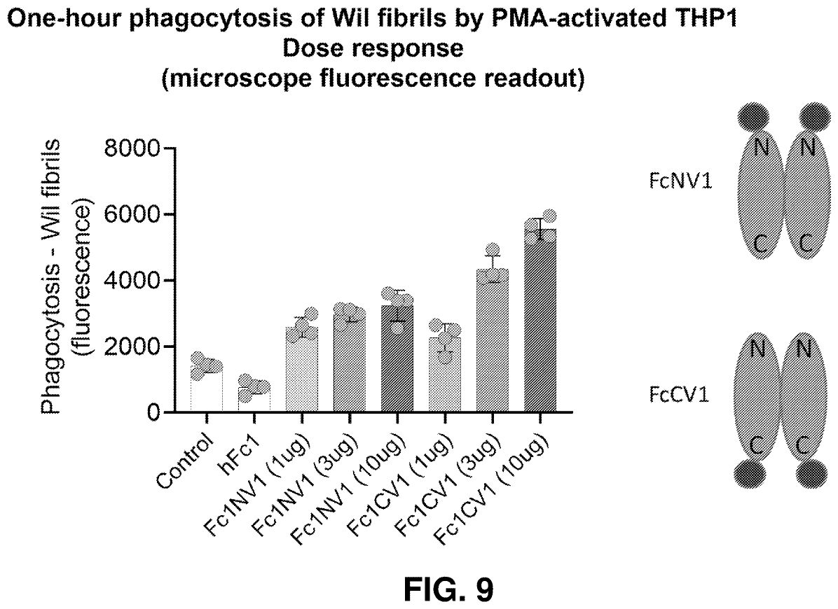

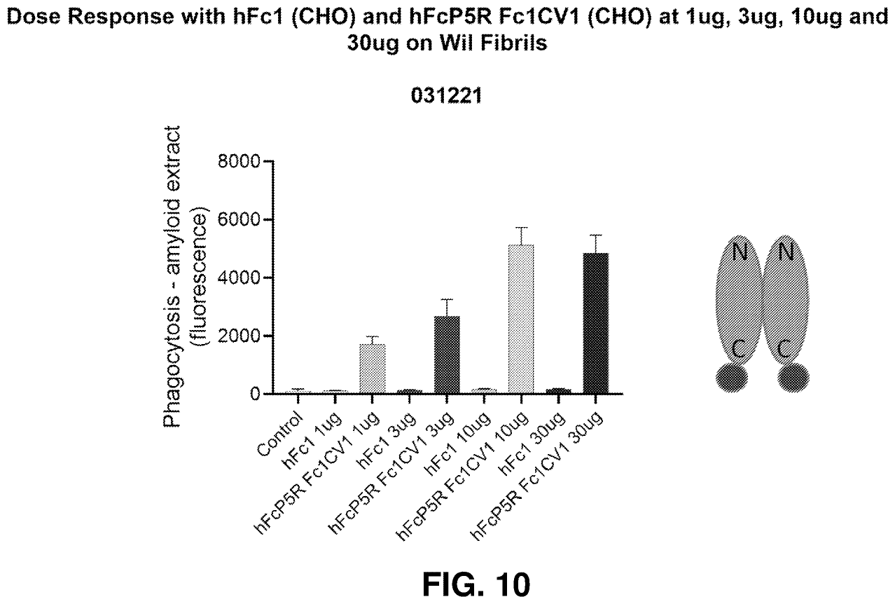

As shown in FIG. 9. Fcp5R CV1 promoted Wil fibril uptake to a greater extent than Fcp5R NV1 did. Without wishing to be bound by theory, the enhanced phagocytic activity induced by Fcp5R CVI is thought to be due to the presence of full-length amyloid-reactive peptide in this variant, relative to the Fcp5R NV1. Further, Fcp5R CV1 exhibited a dose dependent response in Wil fibril uptake, as shown in FIG. 10.

Further, the ability of Fcp5R CV1 to bind rVλ6Wil fibrils was measured, as compared to a human Fc1 control. As shown in FIG. 11, Fcp5R CV1 bound rVλ6Wil fibrils with an EC₅₀ of 2.5 nM, whereas the human Fc1 control did not bind the fibrils.

### Example 4. Design of Further Peptide-Fc Constructs

Further peptide-Fc constructs are contemplated. In one construct, the p5R peptide is fused to the C-terminus of a first and second Fc domain via a flexible, long spacer (GGGGSGGGGS, SEQ ID NO: 16), as shown in the third row of FIG. 1. In another construct, the p5R+14 peptide (an extended p5R variant with 14 more amino acids including an additional four amyloid-binding arginine residues) is fused to the C-terminus of a first and second Fc domain via a short rigid spacer (VSPSV, SEQ ID NO: 15), as shown in the bottom row of FIG. 1. The amino acid sequences of these constructs are provided in Table 4, below.

TABLE 4
Amino acid sequences of
peptide-Fc constructs

| | | SEQ |
|---|---|---|
| | | ID |
| Construct | Amino acid sequence | NO |
| hFc1CV2 | APGGGSVSDKTHTCPPCPAP | SEQ |
| IgG1-Fc | ELLGGPSVFLFPPKPKDTLM | ID |
| p5R, | ISRTPEVTCVVVDVSHEDPE | NO: |
| Flexible | VKFNWYVDGVEVHNAKTKPR | 21 |
| long spacer | EEQYNSTYRVVSVLTVLHQD | |
| | WLNGKEYKCKVSNKALPAPI | |
| | EKTISKAKGQPREPQVYTLP | |
| | PSRDELTKNQVSLTCLVKGF | |
| | YPSDIAVEWESNGQPENNYK | |
| | TTPPVLDSDGSFFLYSKLTV | |
| | DKSRWQQGNVFSCSVMHEAL | |
| | HNHYTOKSLSLSPGKGGGGS | |
| | GGGGSRAQRAQARQARQAQR | |
| | AQRAQARQARQ | |
| hFc1CV3 | APGGGSVSDKTHTCPPCPAP | SEQ |
| IgG1-Fc | ELLGGPSVFLFPPKPKDTLM | ID |
| p5R+14, | ISRTPEVTCVVVDVSHEDPE | NO: |
| Short rigid | VKFNWYVDGVEVHNAKTKPR | 22 |
| spacer | EEQYNSTYRVVSVLTVLHQD | |
| | WLNGKEYKCKVSNKALPAPI | |
| | EKTISKAKGQPREPQVYTLP | |
| | PSRDELTKNQVSLTCLVKGF | |
| | YPSDIAVEWESNGQPENNYK | |
| | TTPPVLDSDGSFFLYSKLTV | |
| | DKSRWQQGNVFSCSVMHEAL | |
| | HNHYTQKSLSLSPGKVSPSV | |
| | RAQRAQARQARQAQRAORAQ | |
| | ARQARQAQRAQRAQARQARQ | |

### Example 5—Biodistribution of ¹²⁵I-Fcp5RCV1 in Mice with Systemic Serum Amyloid Protein A-Associated (AA) Amyloidosis

hFc1CV1 was expressed by transiently transfected CHO cells. The Fc-peptide fusion was purified by Protein A. hFc1CV1 was radiolabeled with iodine-125 by oxidative incorporation into tyrosine side chains. The free radioiodide was separated by size exclusion chromatography and the radiopurity assessed by SDS-PAGE and autoradiography.

Approximately 100 μCi (10 μg reagent) was injected IV into the lateral tail vein of mice with systemic AA amyloidosis or amyloid-free WT mice (as a control). The transgenic AA mice develop systemic amyloid in all organs and tissues but a characterized by severe amyloid in the liver, spleen, and kidneys with only scant deposits seen in the heart. At 1 h, 4 h, 24 h and 48 h post injection the mice (n=4 per group) were euthanized by isoflurane overdose and the SPECT/CT images acquired.

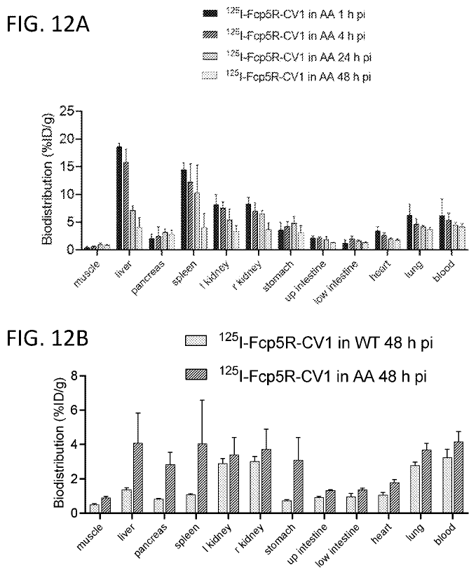

Immediately thereafter, samples or organs and blood were collected for measurement of tissue associated radioactivity as a measure of the biodistribution of the reagents in the organs. This analysis revealed rapid accumulation in amyloid laden organs within 1 h post injection notably in the retention of the 125I-hFc1CV1 in the liver, spleen, kidneys, stomach, and heart with decreasing radioactivity observed in these organs over time (FIG. 12A).

At 48 h post injection there was significant retention of radiolabeled hFc1CV1 in liver, spleen, pancreas, stomach and heart as compared to 125I-hFc1CV1 in WT mice indicating specific binding to a ligand, amyloid in these organs (FIG. 12B).

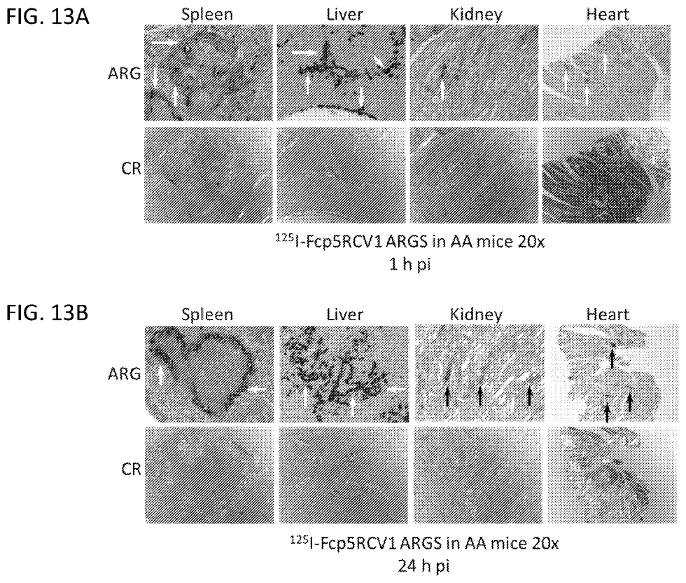

Distribution of the radiolabeled hFc1CV1 in the AA mouse organs was assessed using microautoradiography. Samples of tissue were fixed for 24 h in 10% buffered formalin, embedded in paraffin blocks and 6 μm-thick sections of tissue prepared on glass slides. The slides were exposed to photographic emulsion for three days and then counterstained with H & E. The presence of radioactivity in the tissues was evidenced by the deposition of black silver grains. In all organs evaluated the binding of radiolabeled hFc1CV1 was observed associated with amyloid deposits in the tissue, indicative of specific binding to the pathology (FIG. 13A and FIG. 13B). At 1 h post injection (FIG. 13A) the 125I-hFc1CV1 was already accumulating at specific sites in the tissues, evidenced in the autoradiographs (ARG), which correlated with the presence of amyloid, seen as green/gold birefringence in Congo red (CR)-stained tissue sections. At 24 h post injection (FIG. 13B) the intense and specific amyloid binding of 125I-hFc1CV1 was still evident in the ARGs.

### Example 6—hFc1CV1 Enhances Phagocytosis of Human AL Amyloid Extract In Vitro

hFc1CV1 was expressed by stably transfected CHO cells grown under perfusion culture conditions and purified at day 7. The Fc-peptide fusion was purified by Protein A.

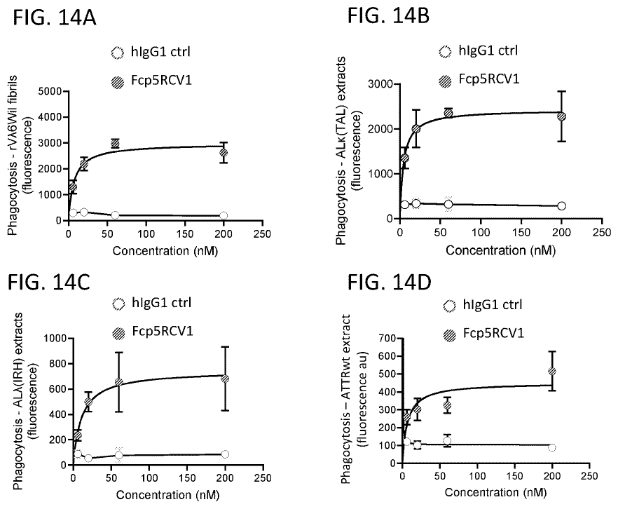

Amyloid-like fibrils (rVλ6WIL), human AL extracts (ALλ or ALκ) and human ATTRwt amyloid extracts were labeled with the pH sensitive dye succinimidyl-pHrodo red fluorophore, for use in an ex vivo phagocytosis assay. Human THP-1 cells were activated by addition of phorbol myristate acetate (PMA) and seeded onto the wells of a 24-well tissue culture plate. A 20-μg mass of amyloid extract was added to the wells with increasing amounts of hFc1CV1 or control hIgG1 antibody (6 nM, 20 nM, 60 nM and 200 nM) and the plates incubated for 1 h at 37 C. The wells were viewed using an inverted fluorescence microscope (Keyance BZ X800) and four digital images (4× objective) captured for each well. The fluorescence in each image was quantified using spectral segmentation and the mean and SD of the four images determined (FIG. 14A-14D).

The results demonstrate that hFc1CV1 enhances phagocytosis of diverse amyloid extracts by activated human THP-1 macrophages in a dose-dependent manner with saturation of the effect at approximately 60 nM for both AL and ATTRwt extracts. The enhancement of fluorescence emission due to increased phagocytosis of the amyloid substrates was significantly greater than the control hlgG1 at all concentrations.

These data demonstrate that opsonization of human amyloid by hFc1CV1 results in significant phagocytosis of amyloid by human macrophages.

### Example 7 Human Plasma, as a Source of Complement, Enhances hFc1CV1 Mediated Phagocytosis of Human AL Amyloid Extract In Vitro

hFc1CV1 was expressed by stably transfected CHO cells grown under perfusion culture conditions and purified at day 7. The Fc-peptide fusion was purified by Protein.

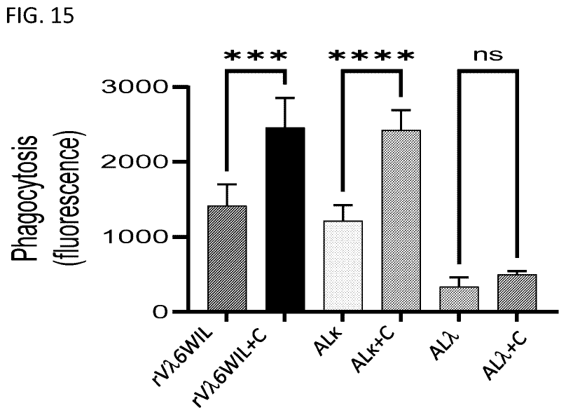

Amyloid-like fibrils (rVλ6WIL) and human AL extracts (ALA or ALK) amyloid extracts were labeled with the pH sensitive dye succinimidyl-pHrodo red fluorophore. Human THP-1 cells were activated by addition of phorbol myristate acetate (PMA) and seeded onto the wells of a 24-well tissue culture plate. A 20-μg mass of amyloid extract was added to the wells with 60 nM hFc1CV1 in the presence or absence of 20% human plasma (as a source of complement). The wells were viewed using an inverted fluorescence microscope (Keyance BZ X800) and four digital images (4× objective) captured for each well. The fluorescence in each image was quantified using spectral segmentation and the mean and SD of the four images determined. The results demonstrate that plasma/complement significantly enhances the efficacy of hFc1CV1 for inducing phagocytosis of human amyloid extracts by activated human THP-1 macrophages (FIG. 15).

These data demonstrate that opsonization of human amyloid by hFc1CV1 in the presence of complement components in plasma results in significantly enhanced phagocytosis of amyloid by human macrophages.

### Example 8 hFc1CV1 Binds Diverse Forms of Amyloid with Subnanomolar Potency

hFc1CV1 was expressed by stably transfected CHO cells grown under perfusion culture conditions and purified at day 7. The Fc-peptide fusion was purified by Protein A.

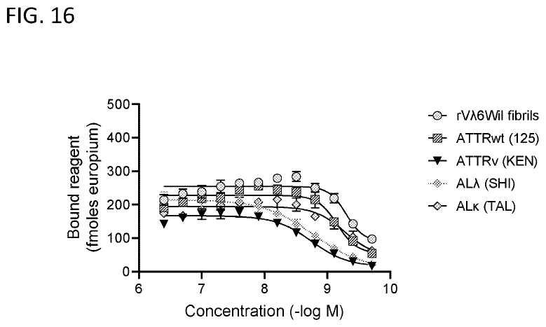

Synthetic amyloid like fibrils (rVλ6WIL) as well as human AL extracts (ALA or ALK) and human ATTRV and ATTRwt amyloid extracts were used as the substrate for binding of hFc1CV1. The Fc-peptide conjugate was added to the wells in 2-fold serial dilution starting at 400 nM. Detection of bound hFc1CV1 was assessed by measuring time-resolved fluorescence, following addition of a biotinylated goat anti-human Fc-reactive secondary antibody and streptavidin-europium conjugate (FIG. 16). The mean and SD of three replicates were calculated and the potency (EC₅₀) was determined following fitting with a sigmoidal 4 PL equation with logarithmic x-axis (Prism) (Table 5).

The estimated potency (EC50) values for the binding of hFc1CV1 to the amyloid substrates ranged from 0.5 nM (for synthetic fibrils) to 1.8 nM got the ATTRv amyloid extract. These data demonstrate that the high affinity binding of hFc1CV1 for synthetic fibrils and human AL and ATTR amyloid extracts.

TABLE 5
EC50

| EC50 (M) | rVλ6Wil fibrils | ATTRwt (125) | ATTRv (KEN) | ALλ (SHI) | ALκ (TAL) |
|---|---|---|---|---|---|
| wxpFcp5RCV1 | 5.0E−10 | 7.0E−10 | 1.8E−09 | 1.7E−09 | 5.3E−10 |

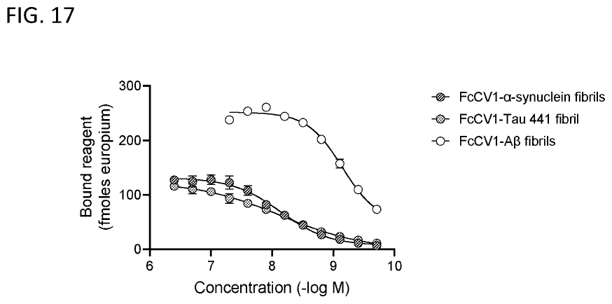

Synthetic amyloid like fibrils (Tau 441, α-synuclein, and Aβ (1-40)) were used as the substrate for binding of hFcCV1. The hFc1CV1 was added to the wells in 2-fold serial dilution starting at 400 nM (50 nM for AB fibrils shown). Detection of bound hFcCV1 was assessed by measuring time-resolved fluorescence, following addition of a biotinylated goat anti-human Fc-reactive secondary antibody and streptavidin-europium conjugate (FIG. 17). The mean and SD of three replicates were calculated and the potency (EC₅₀) was determined following fitting with a sigmoidal 4 PL equation with logarithmic x-axis (Prism) (Table 5). The estimated potency (EC50) values for the binding of hFc1CV1 to the fibrils was 7.3 nM, 7 nM, and 0.7 nM for α-synuclein, Tau 441, and Aβ(1-40) (Table 5).

### Example 8—Binding of Biotinylated hFc1CV1 to Amyloid in Tissue Sections

Formalin-fixed paraffin embedded sections were prepared from tissues containing AL or ATTR amyloid. An additional sample of brain tissue from a patient with Alzheimer's disease was also evaluated. The tissues were stained with biotinylated hFc1CV1 (2 μg/mL in PBS) using standard immunohistochemical methods and visualized following addition of diaminobenzidine. The presence of amyloid in slides from the same tissues was visualized by Congo red fluorescence following staining of the tissues with a solution of alkaline Congo red.

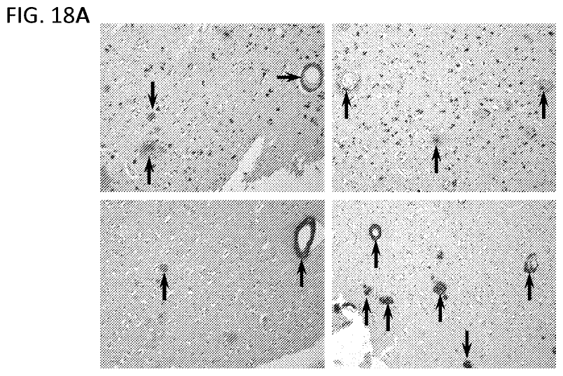

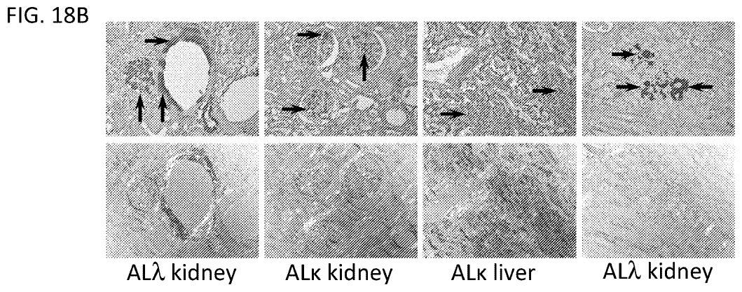

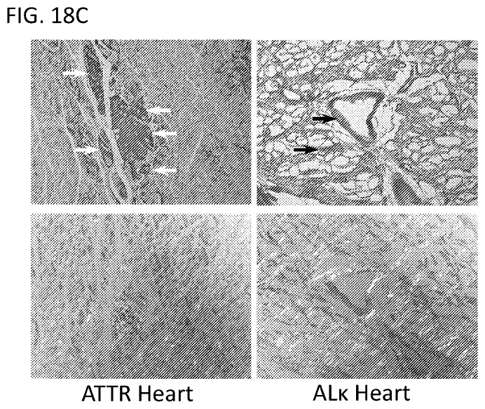

hFcCV1bound specifically to the diffuse and core plaques composed of Abeta amyloid in the brain of a patient with Alzheimer's disease as well as the Abeta amyloid in the vascular walls (FIG. 18A)

Similarly, specific binding with amyloid was observed with AL amyloid deposits in the kidney and liver (FIG. 18B). Specific binding of hFc1CV1 to cardiac amyloid deposits surrounding the cardiomyocytes in two samples of ATTR and AL cardiac amyloidosis (FIG. 18C).

These data demonstrate the specific reactivity of hFc1CV1 with tissue amyloid deposits of varied types in diverse tissues. Thus, the pan amyloid reactivity of hFc1CV1, mediated by the p5R peptide, is evidenced by immunohistochemical staining using tissues from the three most common forms of amyloid-related diseases.

What is claimed is:

1. An amyloid-reactive peptide-Fc fusion protein comprising a first polypeptide and a second polypeptide, wherein the first polypeptide comprises a first amyloid-reactive peptide linked to the N-terminus or C-terminus of a first human Fc domain, wherein the second polypeptide comprises a second amyloid-reactive peptide linked to the N-terminus or C-terminus of a second human Fc domain, and wherein the first and the second human Fc domains form a dimer.

2. The amyloid-reactive peptide-Fc fusion protein of claim 1, wherein the first and second-amyloid reactive peptides are linked to the C-terminus of the first and second human Fc domains.

3. The amyloid-reactive peptide-Fc fusion protein of claim 1 or 2, wherein the first and/or the second amyloid-reactive peptide comprises an amino acid sequence having at least 85% sequence identity to any one of the amino acid sequences set forth as SEQ ID NOs: 1-13.

4. The amyloid-reactive peptide-Fc fusion protein of any one of claims 1-3, wherein the first and/or second human Fc domain is a human IgG1, IgG2, or IgG4 Fc.

5. The amyloid-reactive peptide-Fc fusion protein of any one of claims 1-4, wherein the first and/or second human Fc domain is a human IgG1 Fc.

6. The amyloid-reactive peptide-Fc fusion protein of any one of claims 1-5, wherein the first and/or second human Fc domain comprises an amino acid sequence set forth in SEQ ID NO: 18.

7. The amyloid-reactive peptide-Fc fusion protein of any one of claims 1-6, wherein the first and/or second amyloid-reactive peptide is linked to the first and/or second human Fc domain via a spacer.

8. The amyloid-reactive peptide-Fc fusion protein of claim 7, wherein the spacer is a peptide spacer.

9. The amyloid-reactive peptide-Fc fusion protein of claim 8, wherein the spacer comprises an amino acid sequence set forth in any one of SEQ ID NOs: 14-17.

10. The amyloid-reactive peptide-Fc fusion protein of any one of claims 1-9, wherein the first polypeptide comprises, from N- to C-terminus, a first human Fc domain, a first spacer, and a first amyloid-reactive peptide, and the second polypeptide comprises, from N- to C-terminus a second human Fc domain, a second spacer, and a second amyloid-reactive peptide.

11. The amyloid-reactive peptide-Fc fusion protein of claim 10, wherein the amyloid-reactive peptide comprises the amino acid sequence set forth in SEQ ID NO:2 or SEQ ID NO: 13.

12. The amyloid-reactive peptide-Fc fusion protein of claim 10, wherein the amyloid-reactive peptide comprises the amino acid sequence set forth in SEQ ID NO:2 and the spacer comprises the amino acid sequence set forth in SEQ ID NO:14.

13. The amyloid-reactive peptide-Fc fusion protein of claim 10, wherein the amyloid reactive peptide comprises the amino acid sequence set forth in SEQ ID NO:13 and the spacer comprises the amino acid sequence set forth in SEQ ID NO: 14.

14. The fusion protein of claim 10, wherein the amyloid-reactive peptide comprises the amino acid sequence set forth in SEQ ID NO: 2 and the spacer comprises the amino acid sequence set forth in SEQ ID NO:17.

15. The amyloid-reactive peptide-Fc fusion protein of any one of claims 1-10, wherein
i) the first polypeptide and/or second polypeptide comprises an amino acid sequence set forth in SEQ ID NO: 20;
ii) the first polypeptide and/or second polypeptide comprises an amino acid sequence set forth in SEQ ID NO: 21;
iii) the first polypeptide and/or second polypeptide comprises an amino acid sequence set forth in SEQ ID NO: 22.

16. The amyloid-reactive peptide-Fc fusion protein of claim 15, wherein the first polypeptide comprises an amino acid sequence set forth in SEQ ID NO: 20, and the second polypeptide comprises an amino acid sequence set forth in SEQ ID NO: 20.

17. The amyloid-reactive peptide-Fc fusion protein of any one of claims 1-9, wherein the first polypeptide comprises, from N- to C-terminus, a first amyloid-reactive peptide, a first spacer, and a first human Fc domain and the second polypeptide comprises, from N- to C-terminus a second amyloid reactive peptide, a second spacer, and a second human Fc domain.

18. The amyloid-reactive peptide-Fc fusion protein of claim 16, wherein the amyloid-reactive peptide comprises the amino acid sequence set forth in SEQ ID NO:2 or SEQ ID NO: 13.

19. The amyloid-reactive peptide-Fc fusion protein of claim 17, wherein the amyloid reactive peptide comprises the amino acid sequence set forth in SEQ ID NO:2 and the spacer comprises the amino acid sequences set forth in SEQ ID NO:14.

20. The amyloid-reactive peptide-Fc fusion protein of claim 17, wherein the first and/or second polypeptide comprises the amino acid sequence set forth in SEQ ID NO:19.

21. The amyloid-reactive peptide-Fc fusion protein of any one of claims 1-20, wherein the first and second polypeptides comprise the same amino acid sequence.

22. The amyloid-reactive peptide-Fc fusion protein of any one of claims 1-15 and 17-21 wherein the first and second polypeptide comprise different amino acid sequences.

23. The amyloid-reactive peptide-Fc fusion protein of any one of claims 1-22, wherein the amyloid-reactive peptide-Fc fusion protein binds to rVλ6Wil, Aβ, Aβ(1-40), IAAP, ALκ, ALλ, or ATTR amyloid.

24. The amyloid-reactive peptide-Fc fusion protein of any one of claims 1-23, wherein the fusion protein is conjugated to a detectable label.

25. The amyloid-reactive peptide-Fc fusion protein of claim 23, wherein the detectable label is selected from the group consisting of a fluorescent label and a radioactive label.

26. A pharmaceutical composition comprising the amyloid-reactive peptide-Fc fusion protein of any one of claims 1-24.

27. Nucleic acid(s) encoding the amyloid-reactive peptide-Fc fusion protein of any one of claims 1-25.

28. A vector comprising the nucleic acid(s) of claim 26.

29. A host cell comprising the vector of claim 28.

30. The host cell of claim 29, wherein the host cell is a mammalian cell, optionally a Chinese hamster ovary (CHO) cell.

31. A method of making an amyloid-reactive peptide-Fc fusion protein comprising culturing the host cell of claim 29 or 30 under conditions suitable for expression of the vector encoding the amyloid-reactive peptide-Fc fusion protein.

32. The method of claim 31, wherein the method further comprises recovering the amyloid-reactive peptide-Fc fusion protein.

33. A method of treating an amyloid disease comprising administering a therapeutically effective amount of the amyloid-reactive peptide-Fc fusion protein of any one of claims 1-24 to an individual in need thereof.

34. The method of claim 33, wherein the amyloid related disease is systemic or localized amyloidosis.

35. The method of claim 33, wherein the amyloid related disease is selected from the group consisting of AL, AH, Aβ2M, ATTR, transthyretin, AA, AApoAI, AApoAII, AGel, ALys, ALEct2, AFib, ACys, ACal, AMed, AIAPP, APro, AIns, APrP, Parkinson's disease Alzheimer's disease or Aβ amyloidosis.

36. The method of any one of claims 33-35, wherein the treatment with the amyloid-reactive peptide-Fc fusion protein results in the clearance of amyloid.

37. A method of targeting an amyloid deposit for clearance, comprising contacting an amyloid deposit with the amyloid-reactive peptide-Fc fusion protein of any one of claims 1-25.

38. The method of claim 37, wherein targeting the amyloid deposit for clearance results in clearance of the amyloid deposit.

39. The method of claim 37 or 38, wherein clearance results from opsonization of the amyloid deposit.

40. A method of treating an individual having an amyloid-based disease or suspected of having an amyloid-based disease, comprising:
determining whether the individual has an amyloid deposit by:
detectably labeling the amyloid-reactive peptide-Fc fusion protein of any one of claims 1-25,
administering the labeled amyloid-reactive peptide-Fc fusion protein to the individual,
determining whether a signal associated with the detectable label can be detected from the individual; and,
if the signal is detected, administering to the individual an amyloidosis treatment.

41. A method of identifying an amyloid deposit in an individual, comprising detectably labeling the amyloid-reactive peptide-Fc fusion protein of any one of claims 1-25, administering the fusion protein to the individual, and detecting a signal from the fusion protein.

42. The method of claim 40 or 41, wherein the amyloid-reactive peptide-Fc fusion protein is detectably labeled.

43. The method of any one of claims 31-42, wherein the individual is a human.

44. The amyloid-reactive peptide-Fc fusion protein of any one of claims 1-25, wherein the first polypeptide and the second polypeptide are covalently linked by a disulfide bond in the Fc domain.
